(19) United States
(12) Patent Application Publication
(10) Pub. No.: US 2017/0205426 A1
(43) Pub. Date: Jul. 20, 2017

(54) RAPID MASS SPECTROMETRY METHODS FOR ANTIMICROBIAL SUSCEPTIBILITY TESTING USING TOP-DOWN MASS SPECTROMETRY

(71) Applicant: Thermo Finnigan LLC, San Jose, CA (US)

(72) Inventors: James STEPHENSON, JR., Raleigh, NC (US); Roger GRIST, Ninebanks (GB); William McGEE, Watertown, MA (US); Jason NEIL, Boston, MA (US); David SARRACINO, Andover, MA (US)

(21) Appl. No.: 15/411,814

(22) Filed: Jan. 20, 2017

Related U.S. Application Data
(60) Provisional application No. 62/281,093, filed on Jan. 20, 2016.

(51) Int. Cl. G01N 33/68; C12Q 1/18; G06F 19/18; G06F 19/24

(52) U.S. Cl. CPC G01N 33/6848; G06F 19/24; C12Q 1/18; G06F 19/18; G01N 2333/31; G01N 2333/26; G01N 2333/21; G01N 2333/32; G01N 2800/52

(57) ABSTRACT

Methods and systems for rapid prediction and/or confirmation of antimicrobial susceptibility of a microorganism using top-down mass spectrometry, ion-ion chemistry, and a database with susceptibility, pathogenicity and antimicrobial resistance markers for sample characterization.

### CROSS-REFERENCE TO RELATED APPLICATION

This application claims the benefit of U.S. provisional patent application No. 62/281,093, filed Jan. 20, 2016. The contents of this application are incorporated by reference in their entirety.

### TECHNICAL FIELD

The field of disclosure relates to mass spectrometry and, more particularly, relates to top-down mass spectrometry and using microorganism identification combined with information on susceptibility, typing, pathogenicity and antimicrobial resistance to rapidly and accurately predict antimicrobial susceptibility and pathogenicity.

### BACKGROUND

The purpose of antimicrobial susceptibility testing (AST) is to distinguish between susceptible microbial strains which do not carry clinically significant resistance to antimicrobial agents and microbial strains which have acquired clinical resistance to one or more antimicrobial agents. It is typical practice to test susceptibility against all antimicrobials which have an indicated use against the targeted pathogens. Antimicrobial resistance is now widespread and encompasses practically every available antimicrobial agent. Antimicrobial susceptibility testing is therefore important in clinical practice to establish which antimicrobial agents a microorganism remains susceptible to in order to guide patient therapy. A major limitation with current methodology is reporting is delayed by two to three days from receipt of the specimen into the laboratory. While waiting for an AST result, patients are typically treated empirically based on very limited information which risks treatment failure when the pathogen is resistant to the antimicrobial(s) used for treatment.

A common qualitative AST method is the use of paper disks impregnated with a defined concentration of antimicrobial. The disk is applied to the surface of a plate containing solid nutrient agar upon which a film of liquid containing a dilute microbial culture has been spread over the entire surface and allowed to dry. The plate is incubated during which the antimicrobial diffuses into the media to form a concentration gradient with the highest antimicrobial concentration at the origin. The surface microorganisms grow to form a visible film except where it encounters the antimicrobial at a concentration sufficient to inhibit or kill growth. The more resistant the microorganism, the closer the growth is to the origin. The test is highly reproducible when performed under controlled conditions allowing interpretative criteria to be applied to distinguish between susceptible and resistant isolates. Generally, a resistant microorganism is one that can grow in the presence of a concentration of antimicrobial that would normally be expected to inhibit or kill the microorganism in vivo. The principal of the antimicrobial disk test can be extended to a quantitative measurement by the use of a predefined, continuous, and exponential gradient of antimicrobial concentrations immobilized along a rectangular test strip.

A common method of performing quantitative AST is the broth microdilution susceptibility test. Wells of a microdilution plastic tray contain a doubling dilution series of the antimicrobial and typically many antimicrobials are tested on the same tray. One or more wells lacking the antimicrobial act as the positive control. Nutrient broth containing a suspension of pure microbial culture at a defined viable count is added to each well and the tray is incubated until there is visible growth. The growth in the test wells is compared to the positive control. The end point is defined as the lowest concentration of antimicrobial which suppresses visible growth to give the minimum inhibitory concentration (MIC). This MIC value can be compared to the level achievable at the site of infection to decide whether the organism is resistant or susceptible. Results can be read manually or automatically by measuring turbidity, or by an indirect method such as fluorescence released by the action of culture enzymes on fluorogenic labeled substrates contained in the broth.

Both the paper disk and broth dilution methods require a pure culture and usually 5-24 hour incubation time. Slow growing organisms, such as Mycobacteria, require longer incubation times. The length of incubation can be reduced by periodically monitoring the growth of the culture and reporting results when there is sufficient discrimination between the test wells and positive control. However, some resistance mechanisms are slow to express themselves requiring longer incubation times and may be missed if the reporting process is completed too early. In practice, clinicians want to see results for all antimicrobials that have an indication for use against any type strain or clone. Current rapid susceptibility reporting phenotypic methods miss resistances which develop later in the incubation. Reporting susceptibility against all antimicrobials is therefore delayed until all resistances are detectable which may require incubation periods lasting from overnight to several days.

There are three approaches taken to detecting resistance mechanisms. The most common approach is to deduce the mechanism from the pattern of susceptibility results with different antimicrobials. This approach is used to predict the class of beta-lactamase resistance. For example, for K. pneumoniae, E. coli and K. oxytoca, growth in 4 μg/mL cefpodoxime, 1 μg/mL aztreonam, 1 μg/mL cefotaxime and/or 1 μg/mL ceftriaxone may be indicative of an extended spectrum cephalosporin beta-lactamase (ESBL) producer. See, Table 3A in Clinical & Laboratory Standards Institute (CLSI), MM100-S25, Vol 35, No 3 (2015). Secondly, there are phenotypic tests specific to detection of individual resistance mechanisms such as the modified Hodge test to detect carbapenemase production. A third approach taken to detecting resistance mechanisms is the direct detection of the gene or expressed protein product by either genomic or proteomic methods respectively.

There are a number of published methods aimed at reducing the time for reporting susceptibility information, for example, as summarized in A. van Belkum and W. M. Dunne “Next-generation antimicrobial susceptibility testing” 2013, J. Clin. Microbiol. 51, 2018-2024. All methods require incubation in the presence of antimicrobial and include the following: a) differences in the quantity of ribosomal protein between test and control measured by matrix-assisted laser desorption ionization time-of-flight mass spectrometry (MALDI-TOF) with or without heavy isotope labeling, b) isothermal calorimetry, c) raman and infrared spectroscopy, d) cantilever technology, e) magnetic bead spin, f) flow cytometry, g) bacteriophage amplification, h) microdroplets, or i) real time microscopy to monitor growth of immobilized cells. A number of potential drawbacks to the aforementioned methods which include: a) they require a pure culture so that the specimen must be first plated and incubated to obtain discrete colonies delaying setting up the test by a day, b) requirement for growth in the presence of antimicrobial delays reporting by hours, and c) not all methods are suited for high throughput required for routine clinical testing. As an example, conventional susceptibility testing can miss carbapenemase resistance with false sensitive results with some carbapenem antibiotics. The modified Hodge test is used for reliable detection with detection times of 16-20 hours of incubation or greater for most phenotypic AST/ID systems.

Over the years, widespread use of antimicrobials including broad spectrum agents has encouraged the spread of resistant microbial strains. Dominant clones emerge and spread under this selective pressure. They often combine resistance to multiple antimicrobials with the ability to outcompete other strains. In clinical settings, early resistance detection is imperative to prevent empiric treatment failure and for monitoring the effectiveness of infection control measures aimed at limiting their spread.

There is now more than ever a pressing need for new ways of predicting susceptibility which are sufficiently rapid and accurate to minimize the risk of treatment failure. There remains a need for routine clinical methods for antimicrobial susceptibility testing that are rapid, high throughput, and comprehensive for clinical applications.

### SUMMARY

Disclosed herein are methods and systems which relate, in part, to rapid detection and/or prediction of antimicrobial susceptibility and resistance of a microbial lysate or clinical sample. The methods and systems use top-down mass spectrometry to analyze complex mixtures proteins, peptides, and other biologically relevant molecules to predict or confirm antimicrobial susceptibility of a given microbial isolate or microbial mixture.

In certain embodiments, the present teachings provide a method for predicting antimicrobial susceptibility of a microbe in a sample, the method comprising:
a) disrupting one or more microbes present in a sample to form a fluid microbial extract;
b) separating a soluble protein fraction from an insoluble protein fraction present in the fluid microbial extract to form a liquid solution of the soluble protein fraction;
c) ionizing a stream or flow of the solution of soluble protein fraction to form one or more ionized proteins, wherein the one or more ionized proteins comprises positively charged ions, the positively charged ions comprising a plurality of ion species;
d) analyzing the plurality of ion species with a mass analyzer of a mass spectrometer system to obtain mass spectral data, searching a database containing mass spectral data of known microbial proteins, and identifying at least one microbe from the database based on the determined mass spectral data of the ion species derived from ionization of the proteins from the sample;
e) from a database containing molecular weights and/or sequence data of the analyte compounds associated with antimicrobial susceptibility, selecting a list of analyte compounds whose presence in the identified microbe is predictive of the state of antimicrobial susceptibility for the identified microbe to form a selected analyte database, the list of analyte compounds comprising protein and/or polypeptide compounds;
f) isolating at least a first subset of the plurality of ion species with the mass spectrometer system, each isolated subset of the at least first isolated subset comprising a respective single mass-to-charge (m/z) ratio or range of m/z ratios, wherein the at least first isolated subset is selected based on the microbe identification and the selected analyte database;
g) generating a plurality of first-generation product ions species from each isolated subset of ion species by causing each said isolated subset of ion species to be reacted with reagent anions that, upon reaction, extract protons from each of one or more ion species of said isolated subset of ion species that comprises a protonated molecule of a protein or polypeptide compound whose charge is reduced by the reaction;
h) acquiring at least one mass spectrum using the mass spectrometer system either of some or all of the first-generation product ion species or of a plurality of second-generation product ion species generated by further reaction or fragmentation of the first-generation product ion species;
i) using m/z ratios of the first-generation or second-generation product ion species to search the selected analyte database; and
j) identifying the presence or absence of at least one analyte compound from the sample predictive of the state of antimicrobial susceptibility for the identified microbe.

In some embodiments of this method, the analysis in (d) comprises: i) acquiring one or more mass spectra representative of the plurality of ion species; ii) determining molecular weights for the proteins from the one or more mass spectra; and iii) using the determined molecular weights to search a database containing molecular weights of known microbial proteins and identifying at least one microbe from the database based on the basis of the determined molecular weights of the proteins from the sample. In some embodiments of this method, the analyzing in (d) comprises: i) in a first mass spectrometry step, acquiring one or more first mass spectra representative of one or more of the plurality of ion species; ii) determining molecular weights for the proteins from the one or more first mass spectra; iii) using the determined molecular weights to search a database containing molecular weights of known microbial proteins, and selecting a subset of candidate microbes from the database; iv) in a second mass spectrometry step, selecting one or more precursor ions of the proteins from the of the plurality of ion species and fragmenting the precursor ions by fragmentation means to produce a plurality of MS² product ions; v) using m/z ratios of the one or more precursor ions and/or the plurality of MS² product ions to search a database containing molecular weights of known microbial proteins and product ion m/z values of the known microbial proteins; and identifying at least one microbe from the database based on the basis of the determined molecular weights and product ion m/z values of the ionized proteins from the sample. In other embodiments of this method, the analysis in (d) comprises: i) acquiring one or more mass spectra representative of one or more of the plurality of ion species; and ii) using the acquired mass spectra and a mass spectral deconvolution program that differentiates signals of the mass spectra to search a database containing mass spectra of known microbial proteins and identifying at least one microbe from the database based on the basis of the acquired mass spectra of the ionized proteins from the sample.

In some embodiments of this method, the generating of the plurality of first-generation product ions species from each isolated subset of ion species in (g) is followed by an ion fragmentation reaction of the PTR product species to obtain a plurality of fragment ion species.

In some embodiments, the microbe is identified as a particular subspecies of a microorganism and the list of analyte compounds includes those that are predictive of the state of antimicrobial susceptibility for the particular subspecies. In some embodiments, the microbe is identified as a particular strain of a microorganism and the list of analyte compounds includes those that are predictive of the state of antimicrobial susceptibility for the particular stain. In some embodiments, the microbe is identified as a particular clone of a microorganism and the list of analyte compounds includes those that are predictive of the state of antimicrobial susceptibility for the particular clone.

In certain embodiments, provided herein is a method for predicting antimicrobial susceptibility of a microbe in a sample, the method comprising:
a) exposing a sample containing one or more microbes to culture conditions which can induce production of antimicrobial resistance markers to form an induced sample;
b) disrupting one or more microbes present the induced sample to form a fluid extract;
c) separating a soluble protein fraction from an insoluble protein fraction present in the fluid extract to form a liquid solution of the soluble protein fraction;
d) ionizing a stream or flow of the solution of soluble protein fraction to form one or more ionized proteins, wherein the one or more ionized proteins comprises positively charged ions, the positively charged ion comprising a plurality of ion species;
e) analyzing the plurality of ion species with a mass analyzer of a mass spectrometer system to obtain mass spectral data, searching a database containing mass spectral data of known microbial proteins, and identifying at least one microbe from the database based on the determined mass spectral data of the ion species derived from ionization of the proteins from the induced sample;
f) from a database containing molecular weights of antimicrobial resistance markers expressed by microbes, selecting a list of antimicrobial resistance markers associated with the identified microbe species to form a selected marker database, the list of antimicrobial resistance markers comprising protein and/or polypeptide compounds;
g) isolating at least a first subset of the plurality of ion species with the mass spectrometer system, each isolated subset of the at least first isolated subset comprising a respective single mass-to-charge (m/z) ratio or range or m/z ratios, wherein the at least first isolated subset is selected based on the microbe identification and the selected marker database;
h) wherein the at least first isolated subset of ions are reduced in charge state by: i) at least one proton transfer reaction (PTR) to obtain a plurality of PTR product ion species, or ii) at least one PTR to obtain a plurality of PTR product ion species followed by an ion fragmentation reaction of the PTR product ion species to obtain a plurality of fragment ion species;
i) acquiring at least one mass spectrum using the mass spectrometer system of at least some of the PTR product ion species or at least some of the fragment ion species;
j) using m/z ratios of the PTR product ion species or the fragment ion species to search the selected marker database; and
k) determining the presence or absence of at least one antimicrobial resistance marker from the induced sample, wherein the presence or absence of the identified antimicrobial resistance marker in the induced sample as compared to the microbial sample not induced is predictive of antimicrobial susceptibility for the identified microbe.

In some embodiments, the analyzing in (e) comprises: i) acquiring one or more mass spectra representative of the plurality of ion species; ii) determining molecular weights for the proteins from the one or more mass spectra; and iii) using the determined molecular weights to search a database containing molecular weights of known microbial proteins and identifying at least one microbe from the database based on the basis of the determined molecular weights of the proteins from the induced sample. In some embodiments, the analyzing in (e) comprises: i) in a first mass spectrometry step, acquiring one or more first mass spectra representative of one or more of the plurality of ion species; ii) determining molecular weights for the proteins from the one or more first mass spectra; iii) using the determined molecular weights to search a database containing molecular weights of known microbial proteins, and selecting a subset of candidate microbes from the database; iv) in a second mass spectrometry step, selecting one or more precursor ions of the proteins from the of the plurality of ion species and fragmenting the precursor ions by fragmentation means to produce a plurality of MS2 product ions; v) using m/z ratios of the one or more precursor ions and/or the plurality of MS2 product ions to search a database containing molecular weights of known microbial proteins and product ion m/z values of the known microbial proteins; and identifying at least one microbe from the database based on the basis of the determined molecular weights and product ion m/z values of the ionized proteins from the induced sample. In some embodiments, the analyzing in (e) comprises: i) acquiring one or more mass spectra representative of one or more of the plurality of ion species; and ii) using the acquired mass spectra and a mass spectral deconvolution program that differentiates signals of the mass spectra to search a database containing mass spectra of known microbial proteins and identifying at least one microbe from the database based on the basis of the acquired mass spectra of the ionized proteins from the induced sample.

In some embodiments, the microbe is identified as a particular subspecies of a microorganism and the list of antimicrobial resistance markers includes those that are predictive of the state of antimicrobial susceptibility for the particular subspecies. In some embodiments, the microbe is identified as a particular strain of a microorganism and the list of antimicrobial resistance markers includes those that are predictive of the state of antimicrobial susceptibility for the particular strain. In some embodiments, the microbe is identified as a particular clone of a microorganism and the list of antimicrobial resistance markers includes those that are predictive of the state of antimicrobial susceptibility for the particular clone.

In certain embodiments, provided herein is a method for confirming a predicted antimicrobial susceptibility of an identified microbe in a sample, the method comprising:
a) exposing a sample containing an identified microbe with a predicted antimicrobial susceptibility to culture conditions which can induce production of antimicrobial resistance markers to form an induced sample;
b) extracting, from the induced sample, a liquid solution comprising a mixture of sample-derived proteins and polypeptides;
c) introducing at least a first portion of the liquid solution into an ionization source of a mass spectrometer;
d) generating positively charged ions of the mixture of sample-derived proteins and polypeptides, the positively charged ions comprising a plurality of ion species;
e) isolating at least a first subset of the plurality of ion species, each isolated subset of the at least a first isolated subset comprising a respective single mass-to-charge (m/z) ratio or range of m/z ratios;
f) generating a plurality of first-generation product ions species from each isolated subset of ion species by causing each said isolated subset of ion species to be reacted with reagent anions that, upon reaction, extract protons from each of one or more ion species of said isolated subset of ion species that comprises a protonated molecule of a protein or polypeptide compound whose charge is reduced by the reaction;
g) acquiring at least one mass spectrum using a mass analyzer of the mass spectrometer, either of some or all of the first generation product ion species or of a plurality of second-generation product ion species generated by further reaction or fragmentation of the first-generation product ion species;
h) conducting a search of the at least one mass spectrum of either first-generation or second-generation product ion species for a set of one or more m/z ratios that are diagnostic of at least one marker for the predicted antimicrobial susceptibility of the identified microbe;
i) determining the presence or absence of at least one antimicrobial susceptibility marker from the induced sample; wherein presence of an antimicrobial susceptibility marker in the induced sample as compared to the microbial sample not induced confirms the predicted antimicrobial susceptibility of the identified microbe.

In some embodiments, the at least one marker for the predicted antimicrobial susceptibility is an antimicrobial resistance marker. In some embodiments, a rapid phenotypic antimicrobial susceptibility test was used to predict the antimicrobial susceptibility of the identified microbe prior to (a). In some embodiments, the method further comprises separating a soluble protein fraction from an insoluble protein fraction present in the liquid solution of (b) prior to the introducing of step (c).

In certain embodiments, provided herein is a method for correcting or confirming a predicted antimicrobial susceptibility of a microbe in a sample, the method comprising:
a) disrupting one or more microbes present a sample to form a fluid extract, the sample containing at least one microbe for which a rapid phenotypic antimicrobial susceptibility test (AST) has a predicted an antimicrobial susceptibility;
b) separating a soluble protein fraction from an insoluble protein fraction present in the fluid extract to form a liquid solution of the soluble protein fraction;
c) ionizing a stream or flow of the solution of soluble protein fraction to form one or more ionized proteins, wherein the one or more ionized proteins comprises positively charged ions, the positively charged ion comprising a plurality of ion species;
d) analyzing the plurality of ion species with a mass analyzer of a mass spectrometer system to obtain mass spectral data, searching a database containing mass spectral data of known microbial proteins, and identifying at least one microbe from the database based on the determined mass spectral data of the ion species derived from ionization of the proteins from the sample;
e) from a database containing molecular weights of antimicrobial resistance markers expressed by microbes, selecting a list of antimicrobial resistance markers associated with antimicrobial resistance mechanisms for which the rapid phenotypic AST performs poorly to form a selected marker database;
f) isolating at least a first subset of the plurality of ion species with the mass spectrometer system, each isolated subset of the at least first isolated subset comprising a respective single mass-to-charge (m/z) ratio or range of m/z ratios, wherein the at least first isolated subset is selected based on the microbe identification and the selected marker database;
g) wherein the at least first isolated subset of ions are reduced in charge state by: i) at least one proton transfer reaction (PTR) to obtain a plurality of PTR product ion species, or ii) at least one PTR to obtain a plurality of PTR product ion species followed by an ion fragmentation reaction of the PTR product ion species to obtain a plurality of fragment ion species;
h) acquiring at least one mass spectrum using the mass spectrometer system of at least some of the PTR product ion species or at least some of the fragment ion species;
i) using m/z ratios of the PTR product ion species or the fragment ion species to search the selected marker database; and
j) determining the presence or absence of at least one antimicrobial resistance marker from the sample not predicted by the rapid phenotypic AST and if the at least one antimicrobial resistance marker is present, correcting the predicted antimicrobial susceptibility of the microbe.

In some embodiments for correcting or confirming a predicted antimicrobial susceptibility, the method further comprises, prior to the extracting of (b), exposing the sample to culture conditions which can induce production of antimicrobial resistance markers. In some embodiments, the culture conditions can induce production of antimicrobial resistance markers associated with antimicrobial resistance mechanisms for which the rapid phenotypic AST performs poorly.

In some embodiments of the provided methods, the exposing to culture conditions which can induce production of antimicrobial resistance markers is for about 1 minute to about 30 minutes. In some embodiments, the exposing to culture conditions which can induce production of antimicrobial resistance markers comprises contacting the sample with at least one antimicrobial agent with a concentration below the resistance breakpoint for the at least one antimicrobial agent.

In some embodiments of the provided methods and systems, the separating of the soluble protein fraction is performed using a solid phase extraction device.

In some embodiments of the provided methods and systems, steps are performed using automated instrumentation. For example, in some embodiments of the methods outlined above, steps (a)-(j) or (a)-(k) or (a)-(i) or (b)-(k) are performed using automated instrumentation.

### BRIEF DESCRIPTION OF THE DRAWINGS

To further clarify the above and other advantages and features of the present disclosure, a more particular description of the disclosure will be rendered by reference to specific embodiments thereof, which are illustrated in the appended drawings. It is appreciated that these drawings depict only illustrated embodiments of the disclosure and are therefore not to be considered limiting of its scope. The disclosure will be described and explained with additional specificity and detail through the use of the accompanying drawings in which:

FIG. 1 is a summary of an exemplary top-down rapid mass spectrometry method employed to determine the specific strain of an unknown pathogen.

FIG. 2 is a flow diagram of various data inputs to and outputs from the clinical susceptibility database that links species identification and type/clone information to susceptibility, resistance, pathogenicity and other clinically relevant information. The flow diagram outlines the report process involving predicted patient and infection outcomes, as well as susceptibility information.

FIG. 3 is a typical mass spectrum derived from the methicillin-resistant Staphylococcus aureus (MRSA) strain ATCC 1707 (from American Type Culture Collection) generated from the rapid mass spectrometry process described herein. In the inset is shown the region between m/z 700 and m/z 800 demonstrating the complexity of the sample.

FIG. 4 is a schematic depiction of three different fragmentation pathways and tandem mass spectrometry methods for direct detection of the protein PBP2a derived from clinical MRSA samples for resistance confirmation of Staphylococcus aureus.

FIG. 5 is an electrospray mass spectrum of the intact PBP2a protein derived from a clinical sample.

FIG. 6 shows mass spectra of three of the major N-terminal fragments (top) of PBP2a produced via in-source CID of intact PBP2a and the partial sequence tag (bottom) obtained of the MS³ of the peak found at mass 1304.87 Da (+2 charge state at m/z 653.44).

FIG. 7 is an electrospray mass spectrum of the beta-lactamase SHV-190 derived from the K. pneumoniae strain BAA-2578.

FIG. 8 is a tandem mass spectrum of m/z 997.04 from SHV-190 showing cleavage between leucine and proline to produce the intact C-terminal fragment y₁₁₇ at several charge states.

FIG. 9 is an electrospray mass spectrum of the beta-lactamase KPC-2 derived from the K. pneumoniae strain BAA-1903.

FIG. 10 is an electrospray mass spectrum of the reduced and alkylated form of beta-lactamase KPC-2 derived from the K. pneumoniae strain BAA-1903. Mass addition from alkylation on each cysteine from iodoacetamide is 57.02146.

FIG. 11 is a tandem mass spectrum of the native form of KPC-2 derived from a clinical sample of K. pneumoniae.

FIG. 12 is a tandem mass spectrum of the reduced and alkylated form of KPC-2 derived from a clinical sample of K. pneumoniae.

FIG. 13 is a flow diagram of an exemplary susceptibility pipeline involving the typing process and the two options for confirming susceptibility predictions.

FIG. 14 is an antibiogram table of representative dataset of the type of information that will be utilized for susceptibility prediction. Here the representative strains (Pseudomonas aeruginosa) listed were tested against nine different antimicrobials for three different outcomes: susceptible, intermediate susceptibility, and resistant.

FIG. 15 is an electrospray ionization (ESI) mass spectrum generated from a partially purified protein extract obtained from the bacterium P. aeruginosa.

FIG. 16 is a proton transfer reaction (PTR) product-ion mass spectrum generated by isolating ions of the P. aeruginosa protein extract of FIG. 15 within a 5 Da mass window centered at m/z 990 and reacting the isolated ions with PTR reagent anions. The resulting mass spectrum shows the presence of a 78 kDa protein associated with resistance in P. aeruginosa. The inset of the figure shows an expanded view of resistance protein, the solid diamonds indicate the relevant charge states of the beta-lactamase.

FIG. 17 is an ESI mass spectrum generated from a partially purified protein extract containing beta-lactamase obtained from the bacterium Bacillus cereus.

FIG. 18 is a PTR product-ion mass spectrum generated by isolating ions of the B. cereus protein extract in FIG. 17 within a 5 Da mass window centered at m/z 1101 and reacting the isolated ions with PTR reagent anions. The resulting mass spectrum shows the presence of a 28 kDa protein associated with resistance in B. cereus, the solid diamonds indicate the relevant charge states of the beta-lactamase.

FIG. 19 an electrospray mass spectrum (top) of the recombinant PBP2a protein performed on a linear ion trap mass spectrometer (low resolution spectrum) and a PTR generated spectrum (bottom) of m/z 742.3 for 1 ms. This demonstrates the ability of PTR to separate protein signal from the background noise.

FIG. 20 shows the PTR generated spectrum (top) of FIG. 19 and the concentration of the multiply-charged ion distribution generated via PTR into primarily two charge states using ion parking (bottom).

FIG. 21 shows a PTR generated mass spectrum of the reduced and alkylated form of KPC-2 derived from a clinical sample of K. pneumoniae.

FIG. 22 shows the PTR generated mass spectrum of the PBP2a protein from a MRSA extract with enhanced signal due to the separation of said protein from the background ions at 5 Da window around m/z 775.

### DETAILED DESCRIPTION

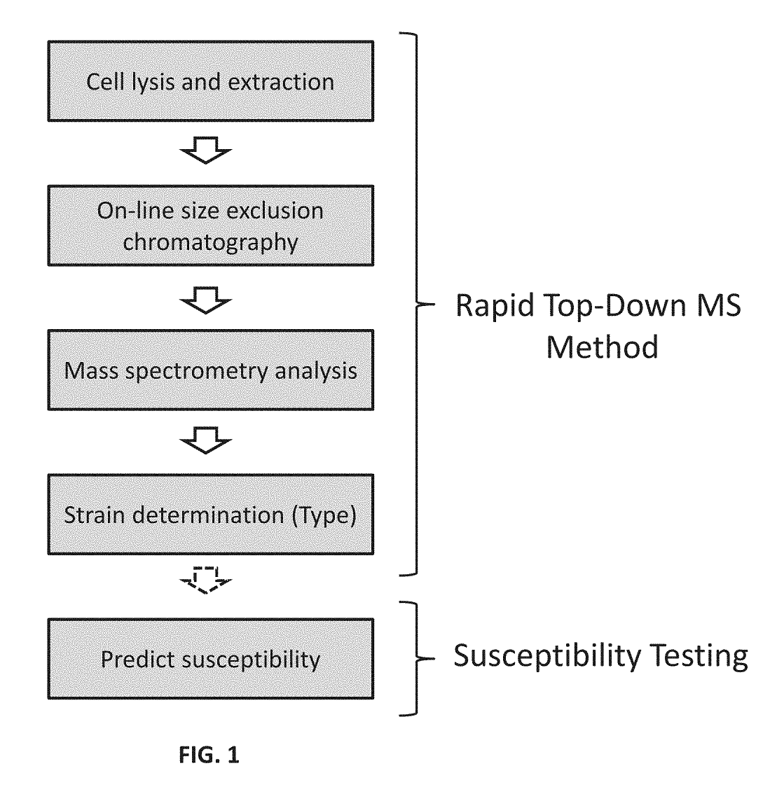

In one aspect, the disclosure provides an alternative to traditional antimicrobial susceptibility testing (AST) methods, namely top-down analysis of intact proteins derived from microbial cells. In general, top-down mass spectrometry interrogates protein structure through measurement of an intact mass followed by direct ion dissociation in the gas phase. Top-down methods allows for analysis of an entire protein molecule without digestion and also allows for low and high mass protein detection. The top-down methods of the present teachings are simple and quick because there is no need for chemical or enzymatic digestion of a sample for the analysis and data processing is accomplished in real time or can use traditional post processing techniques. In some embodiments, analysis of proteins from a microbial lysate is performed via mass spectrometry to identify the microorganism(s) in the lysate at the subspecies type, e.g., strain or clone, level and to identify or characterize protein markers associated with antimicrobial sensitivity and/or resistance. FIG. 1 outlines an exemplary top-down workflow to determine the specific strain of a pathogen and predict its susceptibility where the first four steps in the process occur within about 1 minute. In other scenarios, the first four steps of the workflow of FIG. 1 occur in about 30 minutes or less. Accordingly, in some embodiments, the method from cell lysis to microorganism identification takes between about 1 minute to about 30 minutes, between about 2 minutes to about 5 minutes, or between about 5 minutes to about 20 minutes. In other embodiments, the method from cell lysis to microorganism identification takes about 5 minutes, about 10 minutes, or about 20 minutes. In some embodiments, following the mass spectrometry for microorganism identification, proton transfer reactions (PTR), a type of ion-ion reaction chemistry, is performed on the complex mixtures of ion species generated directly from the ionization of proteins of the typed microbe extract. These rapid separations performed on the millisecond timescale can isolate the known target markers for resistance, virulence, or other proteins associated with relevant clinical outcomes. In certain embodiments, through typing the identified microorganism species, for example, to a sub-species type, e.g., to a strain or a clone of the species, the list of possible antimicrobial sensitivity and/or resistance markers can be restricted for a database search targeted to those linked in the database with the sub-species type. A database search so targeted allows for real-time processing and identification. The methods and systems provided herein provide antimicrobial susceptibility detection, prediction and/or confirmation in a high throughput, comprehensive, and rapid manner.

A database linking microbial species and/or type identification information to all known susceptibility, resistance, and pathogenicity markers is used in the methods and systems provided herein. In some embodiments, such a database may also link species and/or type identification information to known virulence markers. The database may be a single database listing all such information known about an isolate or may be a series of linked databases listing the information. The identification and susceptibility, resistance, pathogenicity and/or virulence information in the database may be based on phenotypic data, proteomic data, and/or genomic data. The database may be built using information known in the art as well as identification and/or susceptibility marker information generated using the methods and systems provided herein. In some embodiments, the database includes molecular weight and/or other mass spectral data information of proteins and polypeptides that can serve to identify microorganisms to, for example, a sub-species, strain, clone, type, and/or serovar. Typically, the database is continuously updated with results from routine clinical testing. In some embodiments, the database includes all known susceptibility, resistance, and pathogenicity markers for local or regional clones and/or types. Testing methods provided herein distinguish between resistant and sensitive strains, as well as information on virulence and pathogenicity.

As the database builds over time, the number of entries for the different species types will increase and can be further extended by including results from other laboratories at a regional, national and international level. The level of resistance (e.g., susceptible or resistant) to each antimicrobial for each microorganism type is continuously updated. Presenting this information at the type and/or clonal level significantly improves empirical prediction and throughput of antimicrobial susceptibility compared to the current practice of calculating the percentage resistance at the species level.

As used herein, microorganism or microbial “typing” or “subtyping” refer to phenotypic and/or genetic analysis of microbial isolates below the species level, for example, to a strain or a clone. As used herein, a microbial “type” refers to a microbial isolate allocated to a named type according to a typing method or assay. Microbial typing and subtyping is performed to generate strain- or clone-specific fingerprints or datasets that can be used, for example, to detect or rule out cross-infections, elucidate bacterial transmission patterns, and/or find reservoirs or sources of infection in human. Resolution varies according to the typing methodology. Typing is described, for example, van Belkum et al. (2007) Clin. Microbial. and Infect. Diseases, CMI, 13 (Suppl. 3), 1-46.

As used herein, “clone” and “clonal complex” refer to isolates of microbial species that are indistinguishable or highly similar in genotype, as identified by using a particular molecular typing approach. Such microbial isolates are assigned as a clone, with the implication they are descended from the same recent ancestor. Clones may be difficult to define with precision since microorgaisms are generally not truly asexual, and recombinational replacements result in diversification of the ancestral genotype of a clone, to produce a cluster of increasingly diverse genotypes called a clonal complex.

The rate at which clonal diversification occurs depends on the extent of recombination, which varies among bacteria, so that some species have rather stable clones (e.g., Salmonella enterica), whereas in other species (e.g., Helicobacter pylori) clones may be so transient that they cannot readily be discerned. Clones and clonal complexes are typically assigned by indexing genetic variation that is selectively neutral, and currently this is achieved, for example, by using multilocus sequence typing. Some species of bacterial pathogens are very diverse, whereas others are genetically uniform, and some are, in essence, a single clone of a mother species that has been raised to species status due to the distinctiveness of the disease it causes (e.g., Yersinia pestis, Salmonella typhi, or Burkholderia mallei). The population structures of bacteria depend on the rate of recombination, and comparative measures of the extent of recombination during clonal diversification can be obtained from multilocus sequence typing data, as can measures of the longer-term impact of recombination. See, for example, Spratt (2004) Methods Mol. Biol. 266:323-352.

Multilocus sequence typing (MLST) is a technique in molecular biology for the typing of multiple loci. The procedure characterizes isolates of microbial species using the DNA sequences of internal fragments of multiple housekeeping genes. See, for example, Spratt (2004) Methods Mol. Biol. 266:323-352. In some instances, microorganism typing may have comparable or better resolution to MLST and be capable of identifying clonal complexes, including international multidrug-resistant high-risk clones. In addition, a clone is defined as isolates that are indistinguishable or highly similar to each other, as identified by using a particular molecular typing approach. See, for example, Spratt (2004) Methods Mol. Biol. 266:323-352. Additionally, an international multidrug-resistant clone may have the following characteristics: international distribution, association with various antimicrobial resistance determinants, the ability to colonize and persist in hosts for long time intervals (>6 months), ability for effective transmission among hosts, enhanced pathogenicity and fitness, and the ability to cause severe and/or recurrent infections.

Acceptable molecular typing procedures for use in conjunction with the provided methods include, without limitation, MLST, pulse field gel electrophoresis (PFGE), PCR typing such as enterobacterial repetitive intergenic consensus (ERIC) or randomly amplified polymorphic DNA (RAPD) methods, multilocus variable-number tandem repeat analysis (MLVA), and whole genome multi-locus sequence typing (wgMLST). Classical MLST schemes typically define seven loci (housekeeping genes), which are sequenced using Sanger technology. Unique sequences for each locus are assigned allele numbers and bacterial strains are identified based on their allelic profiles, which is the combination of the seven allele numbers. As next-generation sequencing is increasingly replacing Sanger sequencing, conventional MLST can be extended to wgMLST. Since many more loci (typically 1500-2500) are considered in wgMLST, a much higher typing resolution can be obtained. wgMLST is based on the concept of allelic variation, meaning that recombinations and deletions or insertions of multiple positions are counted as single evolutionary events. To accommodate the need to maintain a consistent allele assignment for thousands of loci, automated curating tools are available for wgMLST. These molecular typing procedures may also be used to provide information for the database linking microbial identification to susceptibility, resistance, and pathogenicity markers described herein.

The number of mechanisms leading to clinically significant antimicrobial resistance is relatively small and well characterized. Generally, this makes screening for the presence of antimicrobial resistance markers straight forward so long as the marker assay used has adequate sensitivity. The known resistance mechanism information can be used for a targeted search for resistance markers. In certain embodiments, each targeted search may be accompanied with a confirmatory test to verify the test is performing within specification to avoid a false negative result for susceptibility. Preferably, an antimicrobial susceptibility test should be capable of delivering a measurement of susceptibility against all antimicrobials with activity to treat the pathogen.

In some embodiments, samples for analysis using the methods and systems provided herein include, but not limited to, pure or mixed microbial cultures, samples containing a single microorganism type or mixtures of microorganisms, blood culture samples, direct clinical samples (e.g., surface swabs, bodily fluids, etc.), and cultured clinical samples.

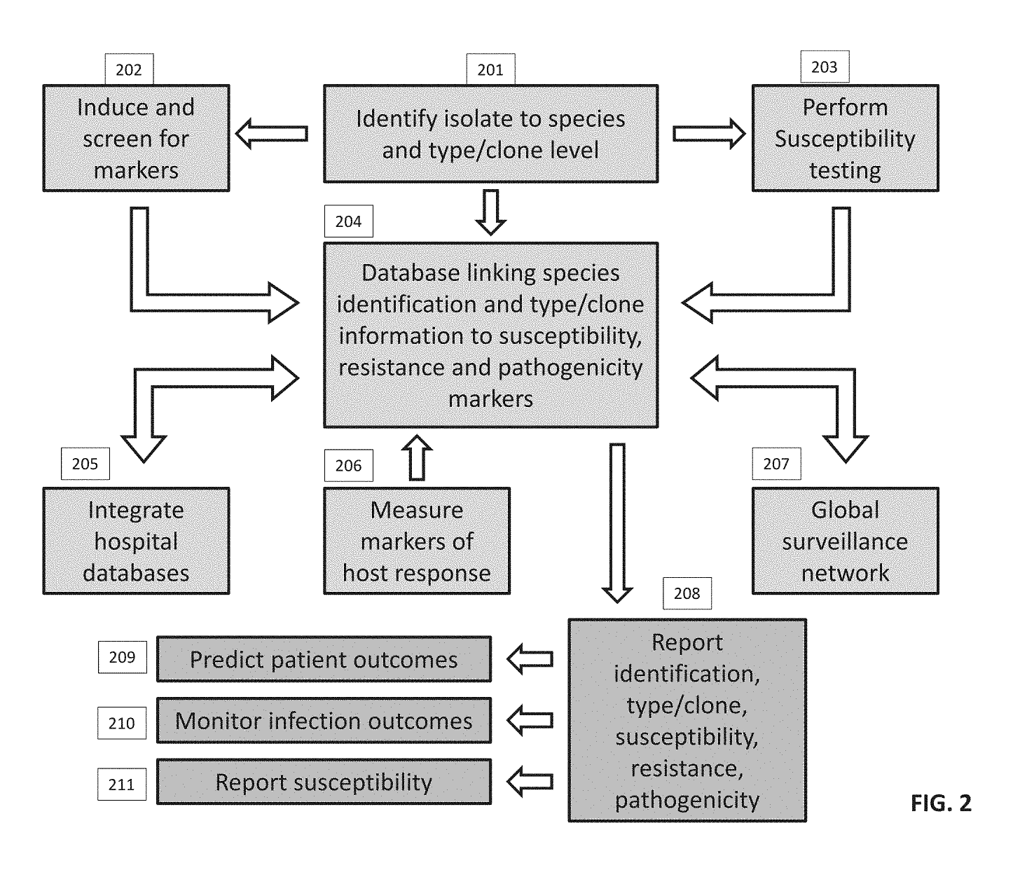

FIG. 2 shows the flow diagram for the identification-susceptibility database process for clonal monitoring. Inputs can be received from a variety of sources including susceptibility testing, other external database at hospitals or other clinical laboratories, genomic sequence information, resistance marker induction processes, host response, or other surveillance networks. The database can include details of pathogenicity markers which have been detected along with the type information and then used to predict pathogenicity and patient outcome. Additionally, host response information (e.g., phenotypic, proteomic, and/or genomic data) can be integrated as well. Mass spectrometry can be used to identify markers indicative of the host response. Such information may be combined with information on the pathogen to better predict clinical outcomes. The flow diagram of FIG. 2 also outlines various report process involving predicted patient and infection outcomes, as well as susceptibility information.

Because a common method, using a limited set of reagents, is performed, the methods of the present teachings are suitable for use within a completely automated system for sample preparation and mass spectrometry. Ideally, these methods may be automated from sample preparation through results reporting. Results may be automatically transferred to a hospital's electronic medical records system where they can be directly linked to patient treatment strategies, insurance, billing, or used in epidemiological reporting. Such an integrated system facilitates epidemiological tracking of an outbreak at the hospital, local, regional, and global levels (see FIG. 2). For high throughput laboratories, multiple systems can be interfaced to a central computer which integrates data from the different instruments from the same or different laboratories prior to reporting. The system can import phenotypic susceptibility data where it can be combined with identification, virulence, pathogenicity, antimicrobial resistance and typing information generated by the methods provided herein.

As used herein, “target markers” include biomarkers of interest, such as proteins and polypeptides, nucleic acids, metabolites, antibiotics and their metabolic derivatives, lipids, or carbohydrates that are directly or indirectly associated with antimicrobial susceptibility, antimicrobial resistance, or microbial pathogenicity. Target markers may also include proteins and polypeptides, nucleic acids, metabolites, lipids, or carbohydrates which are associated with microbial cell growth, microbial cell death or apoptosis, the inhibition of microbial cell growth or static cell growth, and other microbial proteins associated with relevant clinical outcomes.

As used herein, an “antimicrobial’ or “antimicrobial agent” is any substance of natural, semi-synthetic or synthetic origin which inhibits the metabolism and/or growth of a microorganism and can kill it. Antimicrobial agents include antibiotics, bacterial static agents, and bacterial cidal agents.

In therapeutic terms, “susceptible” or “sensitive” to an antimicrobial means that the viability and/or growth of the microorganism is inhibited by a concentration of an antimicrobial agent that typically can be attained in vivo following standard therapeutic doses. A microorganism being in the susceptible category indicates that the antimicrobial agent in question may be an appropriate choice for treating the infection caused by the microbial isolate tested, e.g., the microorganism is likely to respond to treatment with this drug at the recommended dosage.

In therapeutic terms, “resistance” to an antimicrobial agent means that viability or growth of a microorganism is not inhibited by the concentration of an antimicrobial agent that typically can be attained in vivo following standard therapeutic doses. Generally, if a microorganism is resistant to particular antibiotic, that microorganism is expected not to respond to a given drug, irrespective of the dosage and of the location of the infection. Antimicrobials of this category are not the appropriate choice for treating the infection caused by the microbial isolate tested. As used herein, “resistant microbe, microorganism, or bacterium” means an organism which has become resistant to an antimicrobial agent.

The category of “intermediate resistance” is applicable to strains that are “moderately susceptible” to an antibiotic. The intermediate resistance category serves as a buffer zone between susceptible and resistant categories. Intermediate category can be used to indicate a number of possibilities such as, for example, antimicrobials can be used in the body sites where it may be concentrated in the focus of infection (e.g., the urinary tract) or if the high concentration of the antibiotic is used because of its low toxicity.

Susceptibility and resistance to anti-microbial agents are typically expressed as either a concentration or a zone diameter for growth inhibition. Clinical anti-microbial “breakpoints” refer to the minimum inhibitory concentration (MIC) for a given anti-microbial between susceptible and intermediate resistance, between susceptible and resistant and between intermediate and resistant categories. Breakpoints are derived from human clinical studies or from knowledge derived from pharmacodynamic and pharmacokinetic techniques applied to animal models and from in vitro susceptibility data. A large number of national societies and organizations set these break-points using various standards, including the Clinical and Laboratory Standards Institute (CLSI), the European Committee on Antimicrobial Susceptibility Testing (EUCAST), the Food and Drug Administration (FDA) in the US, the Swedish Reference Group for Antibiotics (SRGA), the Japanese Society for Chemotherapy (JSC), etc. (Turnidge, J. et al., Clinical Microbiology Reviews (2007) Vol 20 (3), 391-408.

As used herein, “resistance breakpoint” is the threshold concentration of an antimicrobial agent above which a microbe is considered resistant as defined above.

In some embodiments of the provided methods, high-resolution/mass accuracy single-stage (MS) or multi-stage (MSⁿ) mass spectrometry is used to identify the microorganism(s) in a sample at the genus, species, subspecies, strain pathovar, clone, type or serovar level. In some embodiments, in addition to MS or MSⁿ, multiple stages of proton transfer reaction (PTR) using low resolution scans are used to identify the microorganism(s) in a sample at the genus, species, subspecies, strain pathovar, clone, type, or serovar level. This classification and typing information is then used to simplify the target lists of proteins and markers for analysis to determine clinically relevant results. Methods and systems suitable for such identification of microorganism(s) are described, for example, in U.S. Pat. No. 9,074,236 and in PCT Pat. No. PCT/US2015/040914, each of which is incorporated herein by reference in its entirety.

Types of mass spectrometers suitable for use in the provided methods include, but are not limited to, single and triple quadrupole, two and three-dimensional ion traps, ion cyclotron resonance, Orbitrap detection, time-of-flight, and any hybrid system of the afore mentioned mass analyzers. In various embodiments, methods and systems provided herein may use low or high resolution mass spectrometry instrumentation.

In some embodiments, microbial cells in a sample are disrupted and the soluble proteins extracted and further processed with an on-line clean-up step. Methods that may be used for such an on-line clean-up step include without limitation size exclusion chromatography or rapid solid phase extraction. In some embodiments, following the clean-up step, the microbial protein mixture is directly injected into a mass spectrometer. The identification may be based on acquired mass spectra for pattern matching and/or molecular weight values and fragmentation analysis determined for one or more of the extracted soluble proteins and involves the use of a statistical algorithm(s) to identify the microbe to the type or the clone level. The identification process can occur in real time during the acquisition period or can occur post-acquisition. In some embodiments, the identification uses data dependent mass spectrometry analysis and post-acquisition data-processing as described, for example, in US Pat. Publication No. 2016/0268112, “Methods for Data-Dependent Mass Spectrometry of Mixed Biomolecular Analytes”, and U.S. patent application Ser. No. 15/406,626, “Methods for Top-Down Multiplexed Mass Spectral Analysis of Mixtures of Proteins or Polypeptides” (Yip et al.), filed Jan. 13, 2017, each of which is incorporated herein by reference.

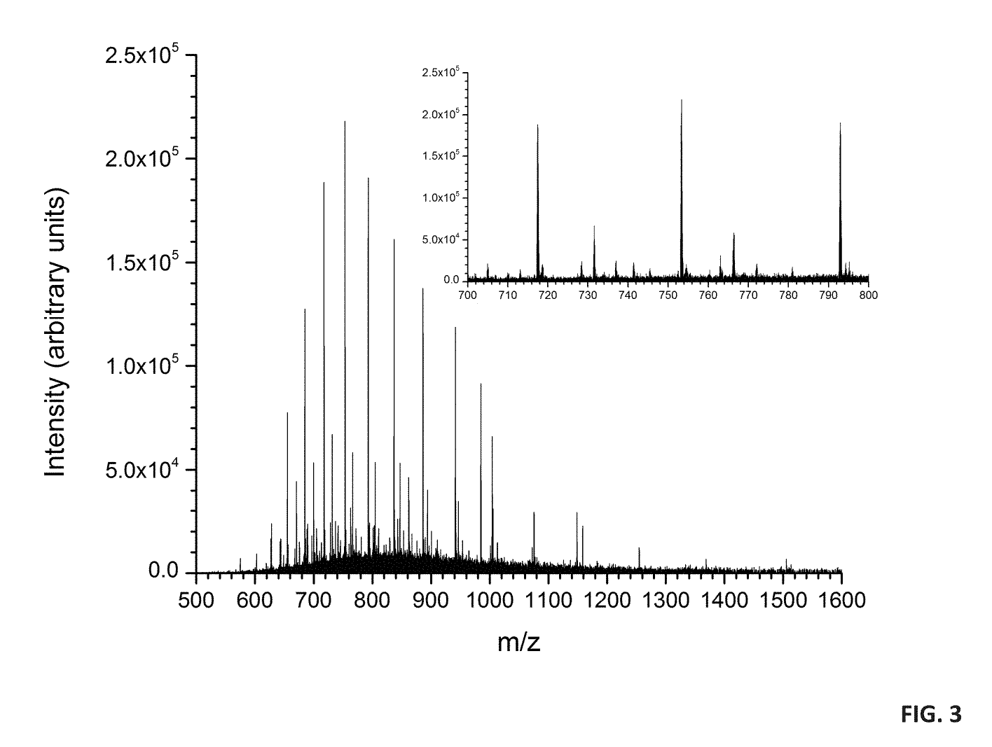

In some embodiments, the identification is performed within a few minutes, for example, less than 30 minutes, within about 20-30 minutes, less than 20 minutes, less than 10 minutes, less than 5 minutes, or within about one minute. In certain instances, tandem mass spectrometry (MS/MS) is used to further confirm the identification of the clone. In some embodiments, the microbial extract is subjected to at least partial chromatographic separation prior to ionization and mass spectrometric analysis. In certain embodiments, the microbial proteins in the extract undergo fast partial liquid chromatographic separation prior to ionization and mass spectrometric analysis. For example, FIG. 3 provides a typical mass spectrum derived from the methicillin-resistant S. aureus (MRSA) strain ATCC 1707 generated from the rapid mass spectrometry identification process. The expanded region shown in the inset demonstrates the complexity of the sample and shows a variety of different signals associated with proteins that can be used to differentiate the pathogen at least to the strain level. In many embodiments, identification of a pathogen to at least the strain level improves throughput of the susceptibility determination approaches described herein.

In certain embodiments, the present disclosure provides an application of ion-ion reaction chemistry in which PTR is employed to determine the presence or absence of resistance markers and the associated antimicrobial susceptibility of complex protein mixtures from microbial and clinical sample extracts. After subjecting a mass-to-charge-restricted subset of ions species derived from ionization of such proteins to PTR, the resulting population of product ions comprises a much simpler population of charge states of lower total charge values (where the words “lower” or “reduced”, in this context, refer to lower or reduced in terms of absolute value) which typically can be readily resolved and assigned to specific protein or peptide ions. In some embodiments, MS/MS may be performed to confirm the protein of interest. This technique can be used to target resistance, pathogenicity and susceptibility markers and markers associated with static cell growth and apoptosis or cell death. Because the PTR product ions represent a smaller subset of multiply-charged species derived from a complex mixture of charge states than the original precursor ions, mass spectral interpretation is greatly simplified and target analysis using tandem mass spectrometry (MS/MS or MS″) can be performed on a single or multiple proteins or other component(s) derived from a complex microbial extract.

The charge-reduced protein and peptide product ions resulting from a given PTR produce mass-to-charge (m/z) values that are greater than those of the original m/z values. For a mixture of protein ions that have the same m/z value but differing mass and charge, the mixture can be separated on the millisecond timescale. This rapid separation process meets the need for the high throughput requirements associated with resistance marker determination and susceptibility testing procedures. Further, these multiply-charged protein ions of the same m/z value with differing mass and charge can be separated from low m/z value background ions derived from small molecules, lipids, solvents, or other interferents that can confound marker confirmation or determination or the identification of other clinically relevant proteins. Multiply-charged ions are therefore separated in time from the background signal thus producing a separated protein or simple mixture of proteins at highly increased signal-to-noise (s/n) ratio. As a result of these two factors, the spectral signatures of the protein/peptide or any other analyte product ions may be significantly separated from a variety of interfering ions. The current disclosure enables both effective (1) non-redundant data dependent mass spectrometry analysis and (2) real time and/or post-acquisition data processing for individual high mass analytes and their mixtures of different complexities. With the embodiments provided herein, data dependent top-down approaches can be applied in real time to target markers of microbial cell growth, stationary phase behavior, or cell death, as well as resistance and pathogenicity markers. In addition, multiple stages of PTR reactions can be performed to separate protein mixtures on low or high resolution instrumentation, such as without limitation a linear ion trap mass spectrometer, in order to simplify and isolate these proteins and other analytes such that target analysis can be performed via MSⁿ analysis. In some embodiments, the advantageous properties of simple PTR reactions may be even further amplified by performing “ion parking” procedures in conjunction with PTR reaction, thus enabling an analyst to at least partially select or control the product-ion charge state distribution that results from the PTR reaction. This real-time ion concentration step may be used to enhance the signal of all associated target markers of any pathogen class.

In some embodiments, PTR involves forming positively-charged ions comprising a plurality of ion species and reacting subsets of the ion species with reagent anions that spontaneously extract protons from each of the one or more ion species, thereby reducing the absolute values of their positive charges. In other embodiments, as an alternative to forming positively-charges ions, PTR involves forming negatively-charged analyte ion species instead. In such cases, the reagent anions or cations are chosen so as to transfer protons to the analyte anions species thereby reducing the absolute values of their negative charges. Control of the PTR processes can be performed manually or automatically in real-time using real-time spectral deconvolution.

In some embodiments, provided herein are methods for predicting antimicrobial susceptibility of a microbe in a sample through generation of a microbial extract, rapid identification of the microbe via high-resolution/accurate mass MS or MSⁿ mass spectrometry, and PTR performed on the complex mixtures used for identification to detect the presence or absence of susceptibility or resistance markers in the microbial extract. In some embodiments, identification of the microbe, for example to the type or strain level, is used to direct or focus the PTR analysis to marker proteins or polypeptides or other biomolecules associated with the identified microbe. PTR is a type of ion-ion reaction chemistry performed in the gas-phase in a mass spectrometer as described herein. PTR methods and systems suitable for use in such marker detection are described, for example, in PCT Pat. No. PCT/US2015/040914, which is incorporated herein by reference.

In some embodiments, PTR is used to identify a microbe at the clone level prior to performing a targeted search for a susceptibility, resistance and/or pathogenicity marker. In other embodiments, screening for a wide range of resistance markers using PTR may be used to identify a microbe at the clone level.

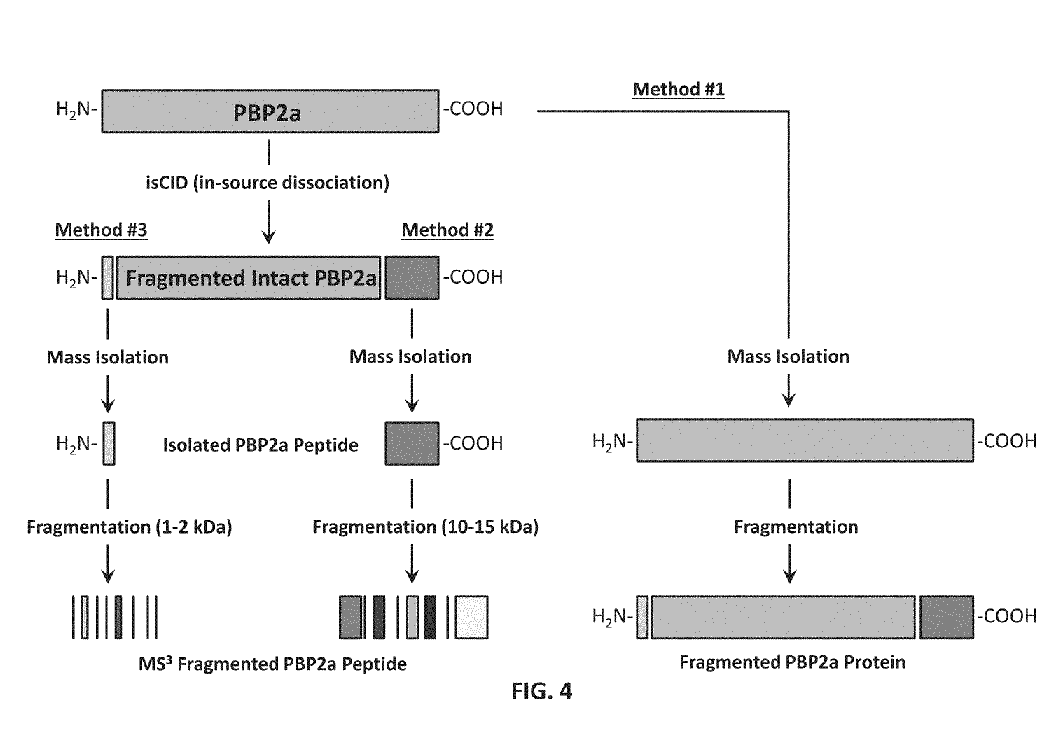

In certain embodiments, the present disclosure provides an application of a variety of different tandem mass spectrometry approaches to determine the presence or absence of resistance markers and the associated antimicrobial susceptibility of complex protein mixtures from microbial and clinical sample extracts. Such tandem mass spectrometry approaches include multiple stages (MS″) of mass spectrometry such as the non-limiting examples shown in FIG. 4. Using analysis of a microbial lysate as an example, FIG. 4 shows three different fragmentation pathways and methods that can be used to identify or confirm the presence of the resistance marker protein PBP2a in the lysate. Method 1 follows a standard route of using targeted MS/MS for confirmation of the PBP2a protein. In contrast, Methods 2 and 3 use an in-source dissociation technique, such as in-source collision-induced dissociation (CID), to first produce either a series of N-terminal fragment ions (Method 2) or C-terminal fragment ions (Method 3) for further analysis. In some embodiments, the in-source dissociation is performed using CID. In other embodiments, dissociation techniques that can be used include without limitation higher-energy collisional dissociation (HCD), electron-capture dissociation (ECD), UV photodissociation, and infrared multiphoton dissociation (IRMPD). As shown in Methods 2 and 3 of FIG. 4, a third stage of tandem mass spectrometry (MS³) including fragmentation of the isolated peptides (e.g., the N-terminal or C-terminal fragment ions) can be used to further improve the signal-to-noise ratio of the peptides (e.g., the N-terminal or C-terminal fragments) to significantly improve detection limits.

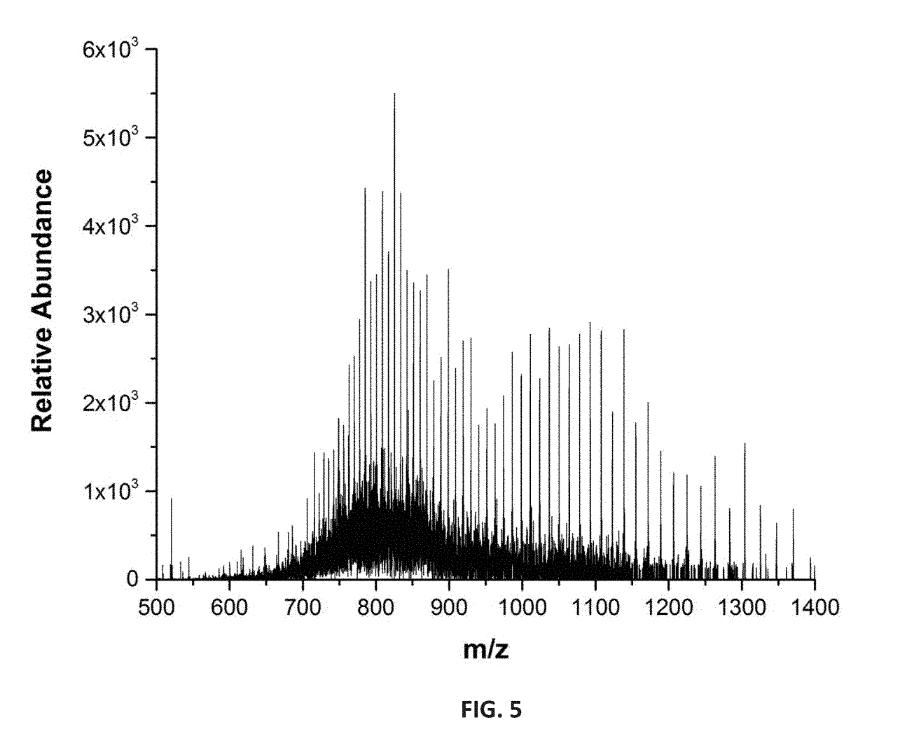

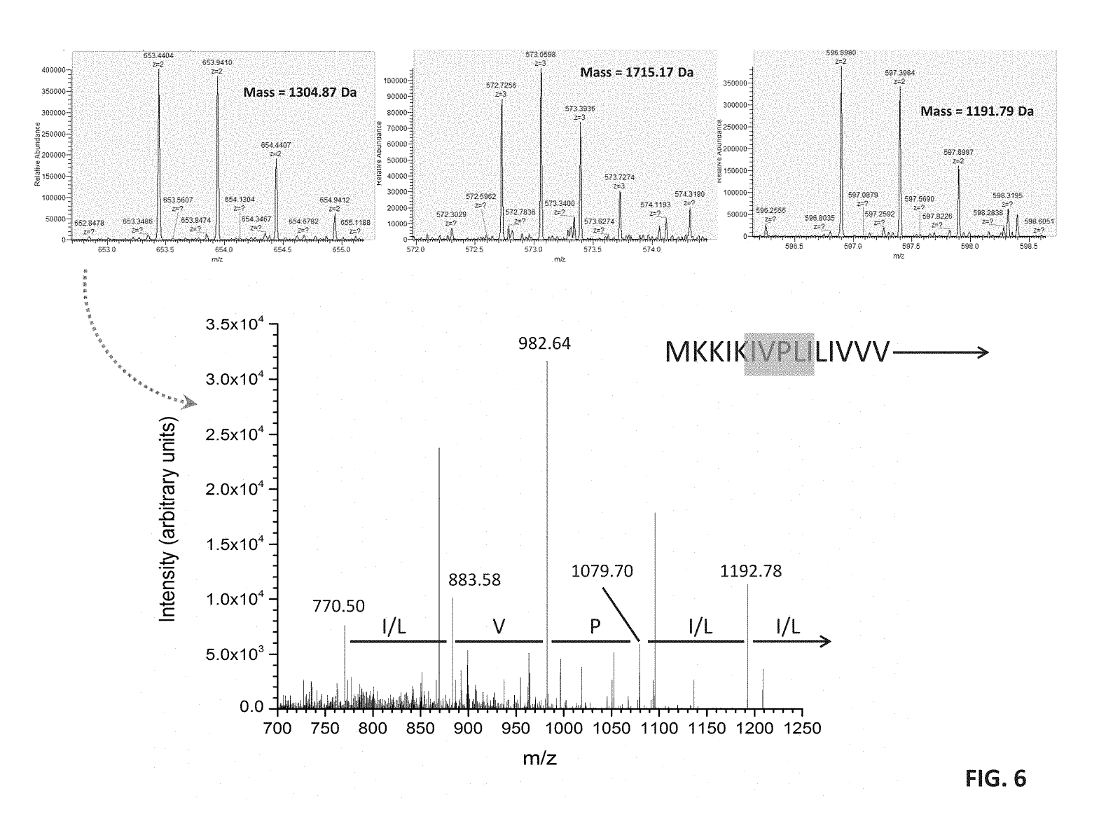

In a preferred embodiment, using Method 3 generates fragments of PBP2a specific to the N-terminus of the protein. FIGS. 5 and 6 illustrate an example of analysis of a MRSA lysate that is performed by the procedure that includes the multiple stages of mass spectrometry as outlined in FIG. 4 Method 3. For this example, clinical MRSA samples were grown on Brilliance™ MRSA 2 Agar plates (Oxoid Limited, Thermo Fisher Scientific) according to according to manufacturer instructions. In FIG. 5 is shown an electrospray mass spectrum of intact PBP2a from a lysate of the MRSA strain which illustrates essentially the starting point for the methods shown in FIG. 4. FIG. 6 illustrates the MS3 analysis of the N-terminal sequence of PBP2a using method 3 of FIG. 4. The top portion of FIG. 6 shows three of the major N-terminal fragments of PBP2a produced via the in-source CID process on intact PBP2a. The lower portion of FIG. 6 shows the partial sequence tag obtained of the MS³ of the peak found at mass 1304.87 Da (+2 charge state at m/z 653.44). The identified sequence tag IVPLI is highlighted in the data at the lower portion of FIG. 6.

The N-terminal portion of PBP2a serves as a membrane anchor for the protein and has a highly conserved amino acid sequence across PBP2a variants, MKKIKIVPLILIVVV. The highly conserved sequence of the PBP2a N-terminus can be used to detect a variety of different relevant clinical variants of the protein.

Accordingly, in some embodiments, tandem mass spectrometry including multiple stages (MSⁿ) of mass spectrometry may be performed to confirm the protein of interest from a complex microbial extract. This technique can be used to target resistance, pathogenicity and susceptibility markers and markers associated with static cell growth and apoptosis or cell death. In some embodiments, such tandem mass spectrometry methods are of particular use when targeting the isolation and further fragmentation of a particular peptide fragment. In some embodiments, targeting the N-terminal fragment ion from PBP2a for further fragmentation and subsequent analysis can be used to detect a PBP2a protein in lysate from a variety of different relevant clinical variants.

In some embodiments, provided herein are methods for predicting antimicrobial susceptibility of a microbe in a sample through generation of a microbial extract, rapid identification of the microbe via high-resolution/accurate mass MS mass spectrometry including an in-source dissociation step performed on the complex mixtures used for identification, followed by targeted MS' mass spectrometry to detect the presence or absence of susceptibility or resistance markers in the microbial extract. In some embodiments, identification of the microbe, for example to the type or strain level, is used to direct or focus the MSⁿ mass spectrometry and analysis to marker proteins or polypeptides or other biomolecules associated with the identified microbe.

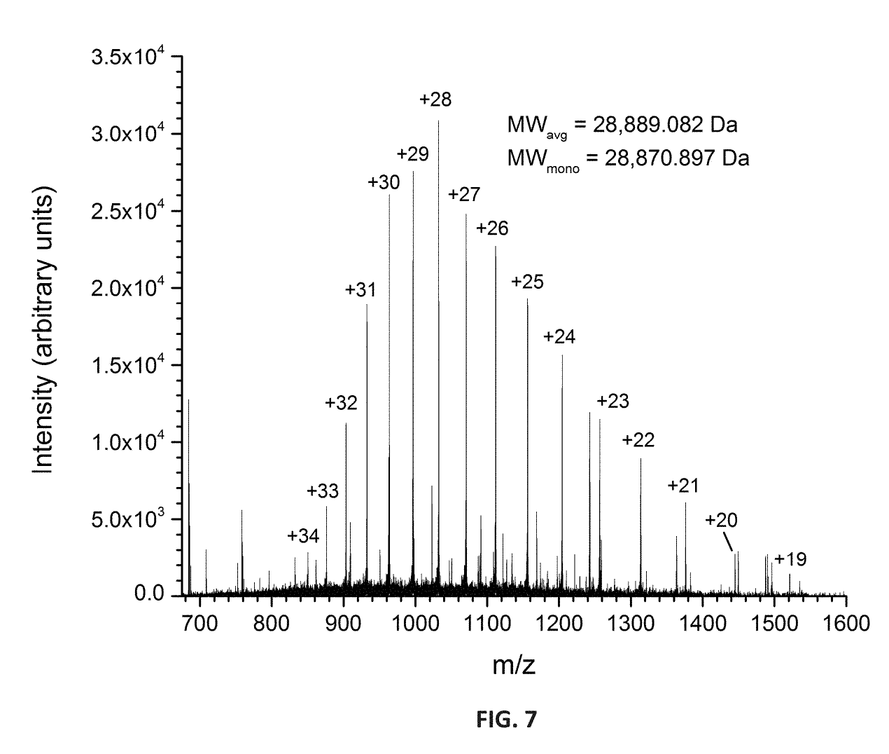

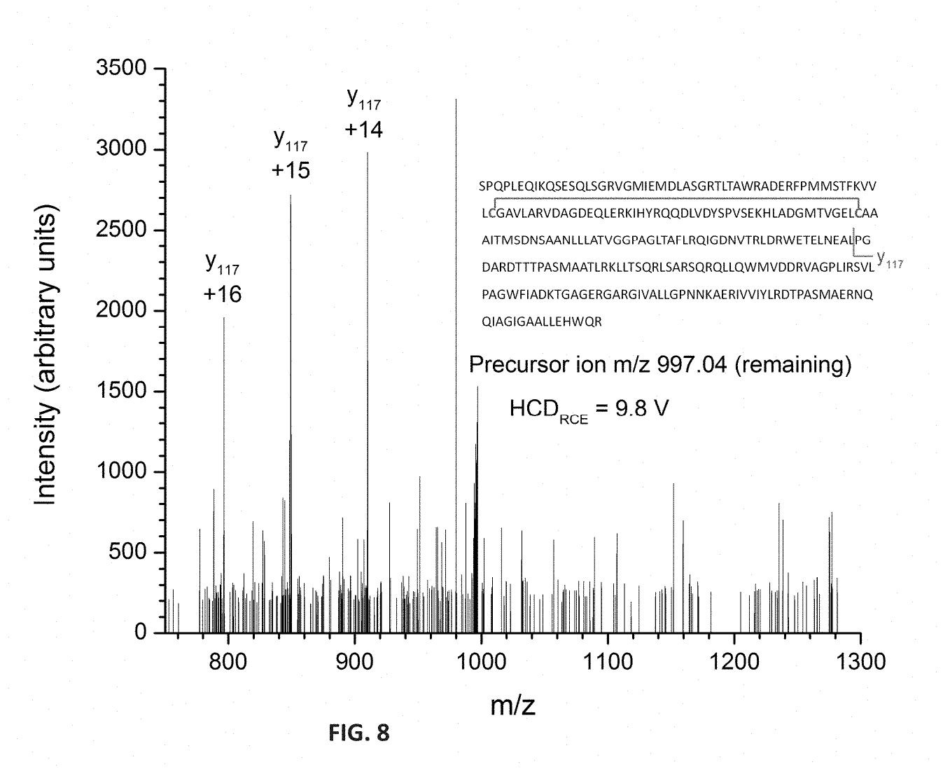

FIGS. 7 and 8 illustrate an example of analysis of Klebsiella pneumoniae extract that was performed by a MSⁿ mass spectrometry method provided herein. For this example, a sample the multiple resistant strain of K. pneumoniae BAA-2578 was cultured and an extract prepared. FIG. 7 depicts the electrospray mass spectrum of the SHV-190 beta-lactamase present in the K. pneumoniae extract. To confirm the presence of this SHV-1 variant, tandem mass spectrometry was used. For this type of beta-lactamase, there are typically two cysteine amino acids present that form a disulfide bridge to help stabilize the protein as it typically operates in the periplasm of Gram negative pathogens. For example, see the protein sequence in FIG. 8. This disulfide bridge can directly influence the fragmentation associated with any protein as the linkage can limit the amount of fragmentation observed in HCD and other CID mass spectra. FIG. 8 depicts the tandem mass spectrum using HCD of m/z 997.04 from SHV-190 with multiple charge states off the same major fragment ion y117, a cleavage between leucine and proline to produce the intact C-terminal fragment indicated in FIG. 8.

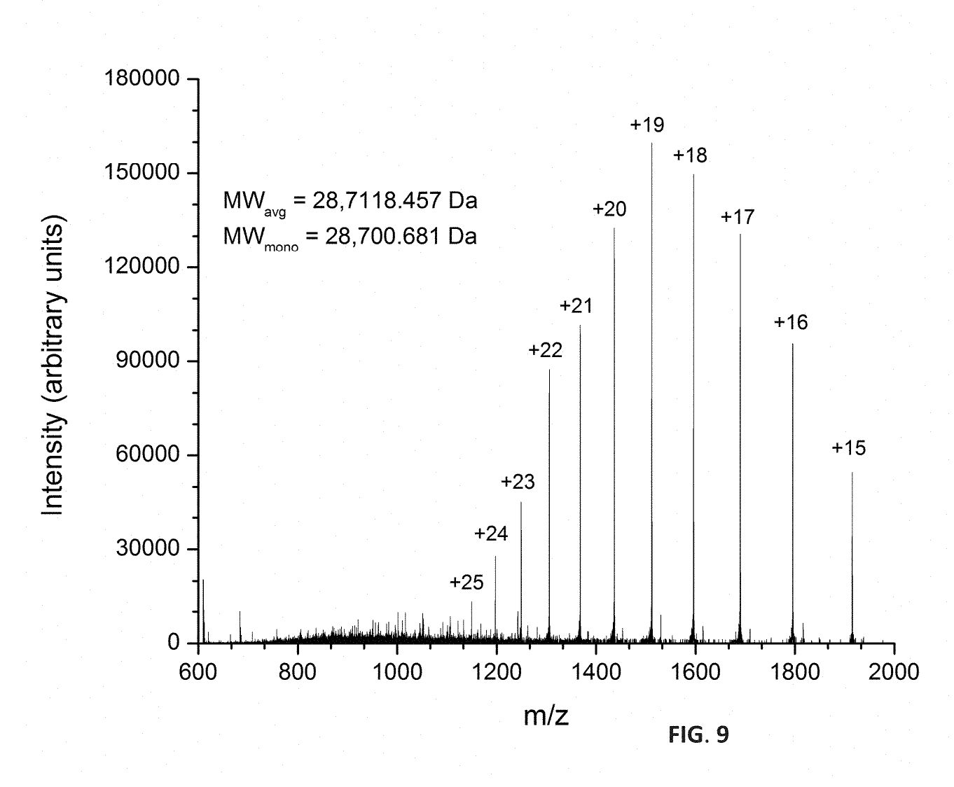

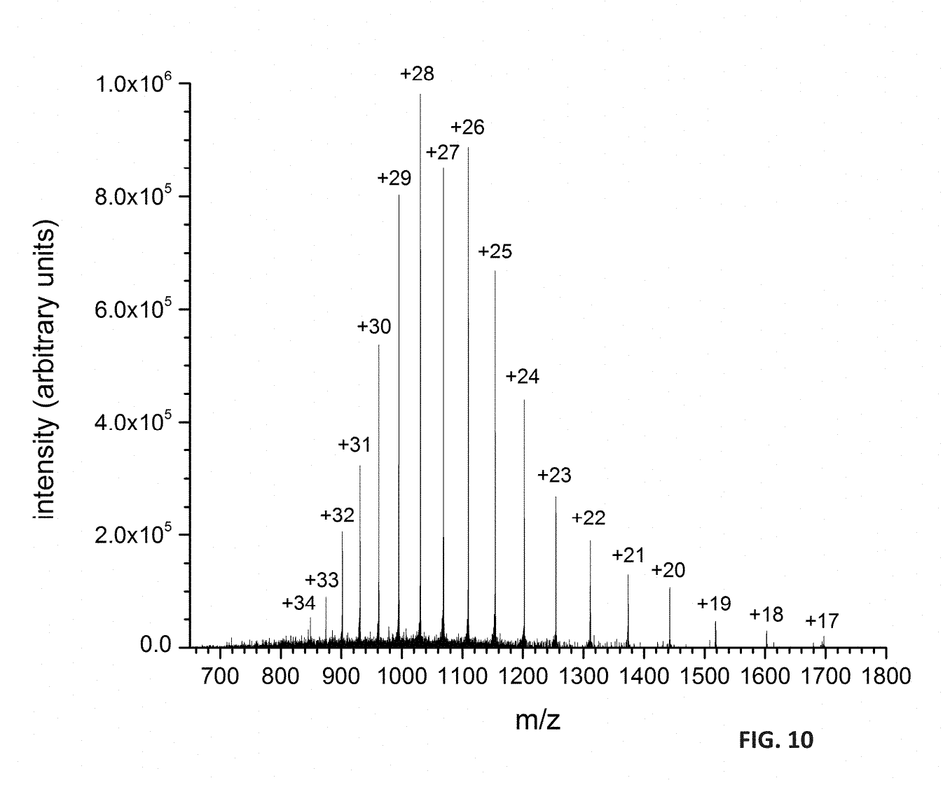

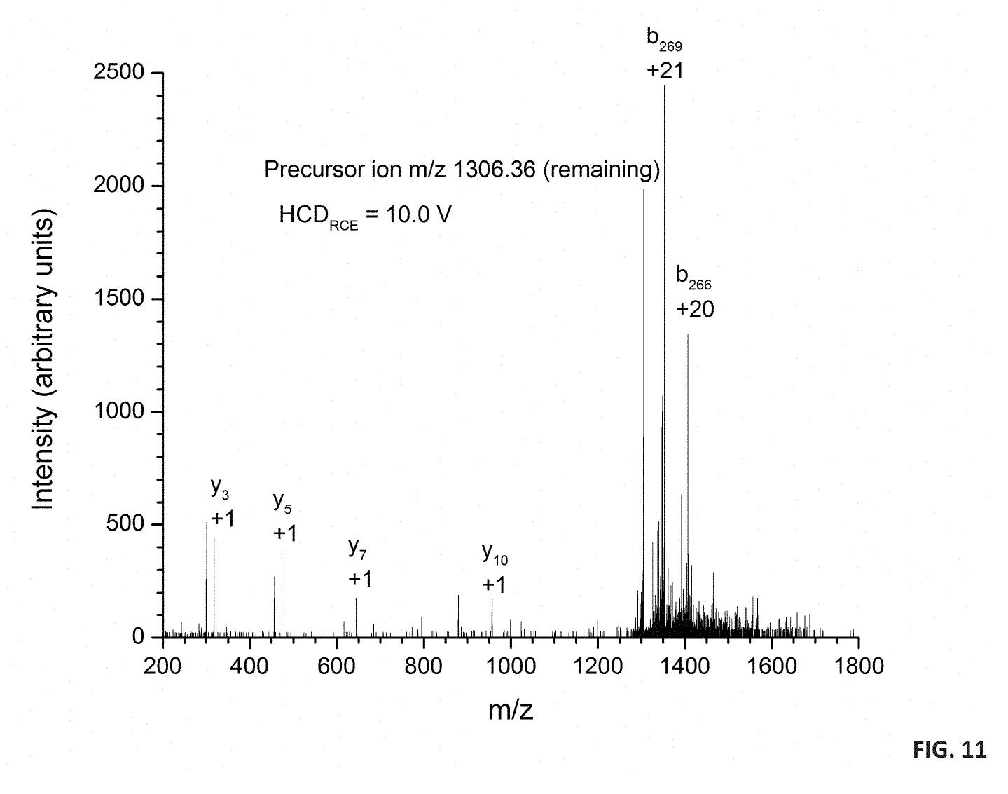

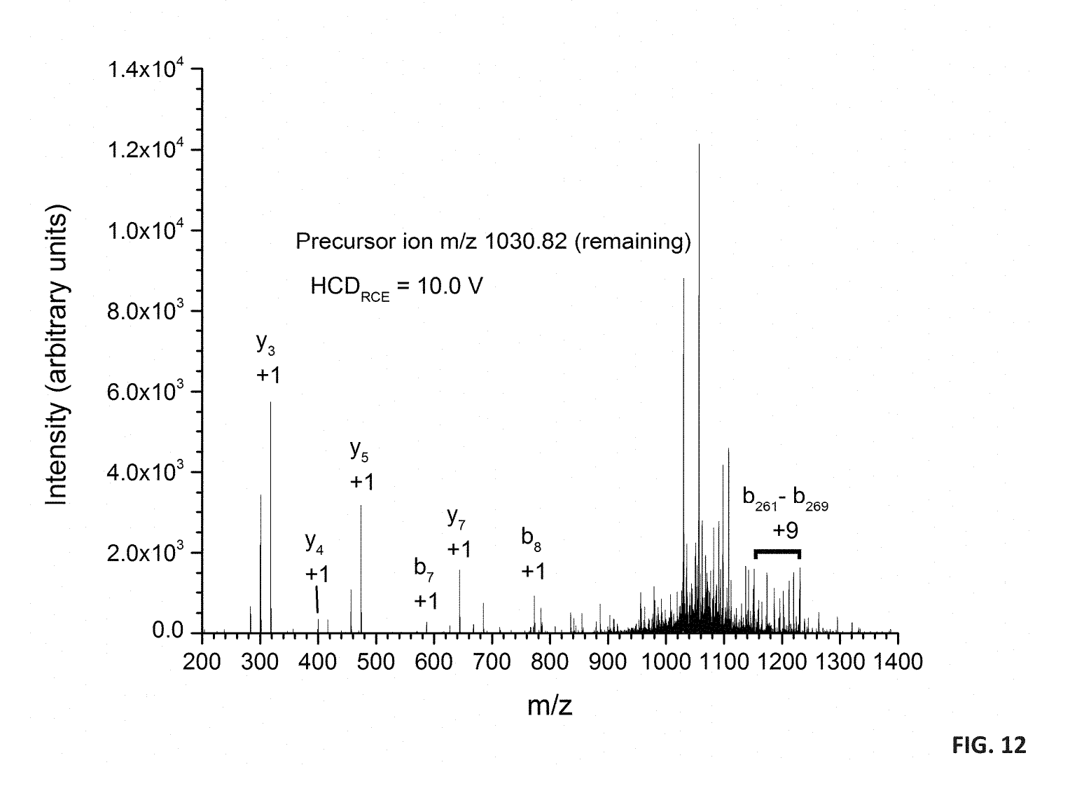

In another example, a similar observation can be made with the beta-lactamase KPC-2. FIGS. 9-12 illustrate an example of analysis of Klebsiella pneumoniae extract that was performed by a MS' mass spectrometry method provided herein. For this example, a sample the clinical strain of K. pneumoniae BAA-1903 was cultured and an extract prepared. FIG. 9 depicts the electrospray mass spectrum of KPC-2 derived from the K. pneumoniae extract. Due to the presence of a disulfide bond in the active form of the protein, the charge state distribution shown is shifted to lower charge states or higher m/z values. FIG. 10 depicts the electrospray mass spectrum of the reduced and alkylated from of KPC-2 from the K. pneumonia extract with the corresponding charge state shift to high charge states and lower m/z values. This analysis was performed to verify that the predicted disulfide bridge exists in the expression of active KPC-2 in a clinical sample. To further confirm the identity of the protein, tandem mass spectrometry using HCD procedures were carried out on both the active and the reduced and alkylated versions of the protein. FIG. 11 depicts the tandem mass spectrum of the native form of KPC-2 from the K. pneumonia extract. FIG. 12 depicts the tandem mass spectrum of the reduced and alkylated for of KPC-2 from the K. pneumonia extract. As shown in FIGS. 11 and 12, the fragmentation of the reduced and alkylated version of the protein is much more extensive than that observed in the native disulfide linked resistance protein. The reduce and alkylated form of the protein was used to verify the presence of a disulfide bond in the KPC-2 resistance marker.

Preferably, an antimicrobial susceptibility test should be capable of delivering a measurement of susceptibility against all antimicrobials with activity against a pathogen, at a cost and throughput capable of matching current methods.

In some embodiments, a sample of the microbial culture or biological sample is exposed to conditions that induce antimicrobial resistance in the microorganisms prior to testing. In other embodiments, a sample of the microbial culture or biological sample is tested without intentionally inducing antimicrobial resistance in the microorganisms. In certain embodiments, many resistance mechanisms may not have to be induced in order to detect the target proteins of interest. For example, we have observed that the production of PBP2a from MRSA samples varies in its baseline expression without induction. In examining expression of carbapenem-resistant Enterobacteriaceae (CRE)-related resistances, several strains of Klebsiella pneumoniae that contained different combinations of the resistance marker beta-lactamase were tested to determine the effect on detection for induced and non-induced samples. Table 1 lists various resistant and susceptible strains of K. pneumoniae which were tested with 0.5 mM of ertapenem as an inducer. In these experiments, bottom-up mass spectrometry was used to measure sequence coverage of two representative beta-lactamases, KPC-2 and CTX-M15. The percent sequence coverage observed is an approximate measure of the amount of a given protein present in any given extract. The higher the percent sequence coverage observed, the higher the level of that given protein in an extract. For the cases of KPC-2 and CTX-M15 induced and non-induced in the variety of different strains, the percent sequence coverage observed was the same regardless of induction. These results indicate that for these resistances that induction is not necessary to confirm the presence or absence of CRE targets.

TABLE 1
Klebsiella pneumoniae beta-lactamase levels for
induced and non-induced cultures.

| | | | KPC-2 | CTX-M15 |
|---|---|---|---|---|
| | | | Sequence | Sequence |
| Activity | Strain | Induced | Coverage | Coverage |
| Susceptible | 13883 | No | not detected | not detected |
| Susceptible | 13883 | Yes | not detected | not detected |
| Resistant | BAA-1903 | No | 72% ± 2% | not detected |
| Resistant | BAA-1903 | Yes | 73% ± 0% | not detected |
| Resistant | BAA-1905 | No | 83% ± 0% | not detected |
| Resistant | BAA-1905 | Yes | 85% ± 5% | not detected |
| Resistant | BAA-2473 | No | not detected | 70% ± 2% |
| Resistant | BAA-2473 | Yes | not detected | 70% ± 0% |
| Resistant | BAA-2578 | No | not detected | 68% ± 0% |
| Resistant | BAA-2578 | Yes | not detected | 60% ± 2% |

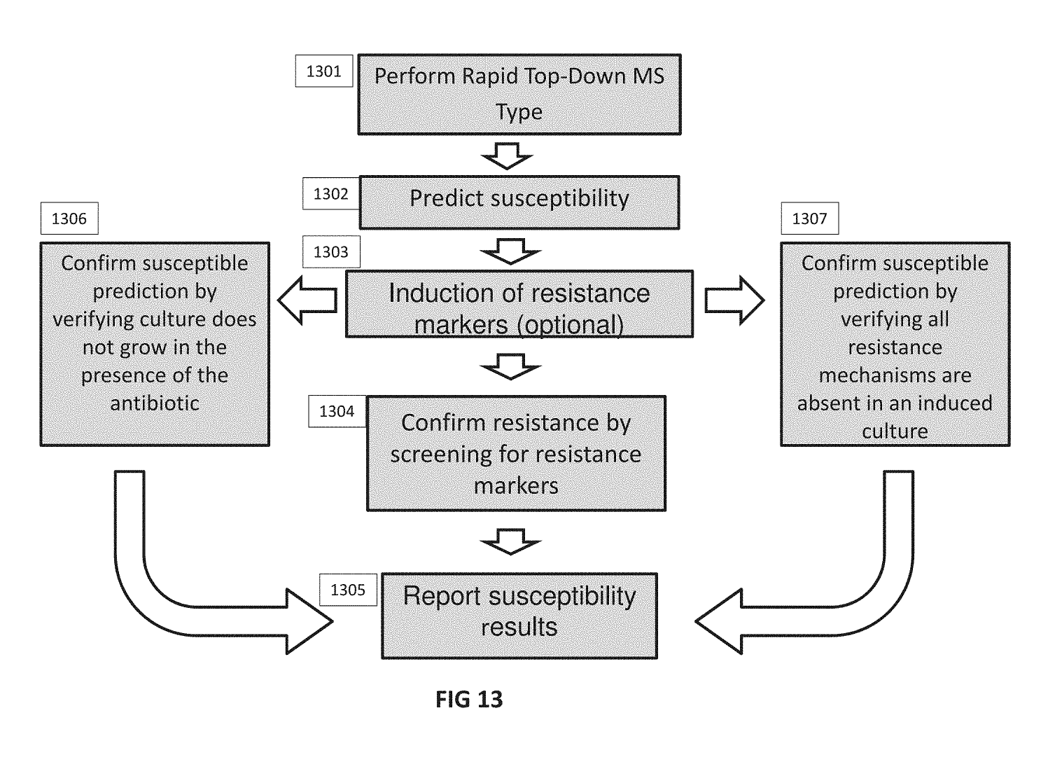

FIG. 13 schematically illustrates a susceptibility pipeline with various aspects involving microbial typing in conjunction with susceptibility prediction and/or confirmation. In some embodiments, steps 1301 and 1302 (FIG. 13) for rapidly predicting susceptibility use rapid top-down mass spectrometry processes described herein to first identify the microorganism(s) in the sample to the type level and then, using the type determination to create a targeted marker database, to confirm the susceptibility, resistance and/or pathogenicity marker(s) for the identified microbe. As described herein, in some embodiments, the 1301 typing step can take about 1 minute and in other embodiments it can take about 1-30 minutes, about 5 minutes, about 10 minutes, or about 20 minutes.

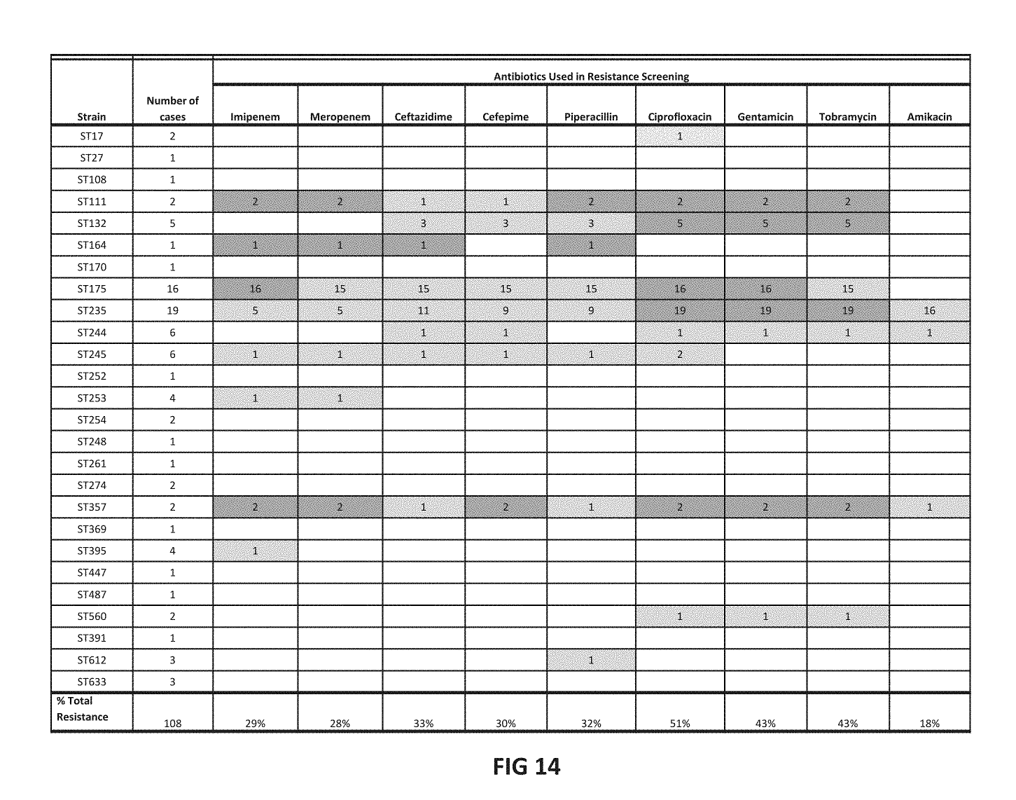

In some embodiments for predicting antimicrobial susceptibility, the rapid mass spectrometry method is used to identify the microorganism to the sub-species level and, using the sub-species determination, an antibiogram based on local isolates is used to predict susceptibility. An antibiogram is a collection of data usually in the form of a table summarizing the percent of individual microbial pathogens susceptible to different antimicrobial agents. FIG. 14 provides an exemplary antibiogram of representative strains of P. aeruginosa tested against nine different antimicrobial agents. FIG. 14 is a summary of resistance screening for 26 of the 42 strains of P. aeruginosa isolated from a total of 108 blood stream infections in the Czech Republic (Nemec et al. (2010) Research in Microbiology 161:234-242). The bottom row in FIG. 14 lists the percentage of the 108 isolates resistant to each of the antibiotics. The antibiogram of FIG. 14 lists the number of cases observed over a year for each identified strain listed. The white shaded blocks refer to where all the P. aeruginosa strains were susceptible to the antibiotic, light gray shading refers to where some isolates displayed resistance, and dark gray indicates when all isolates were resistant. Strains ST175 and ST235 are examples of dominant clones where there are higher frequency of resistance against many antibiotics. The clonal antibiogram information can also be used to guide confirmatory testing.

Presently, laboratories report their antibiograms at the species/genus level. The selection criteria, however, is too wide pulling in resistant and sensitive strains to give a blended average. This is equivalent to the percent total resistance shown in the bottom row FIG. 14. The ability to map the data at a strain or clone level, as provided by the methods and systems described herein, enables the resistant strains to be pulled away from the sensitive ones making prediction of susceptibility more meaningful and useful.

In some embodiments for predicting susceptibility of an identified microorganism or a microorganism of suspected identity in a sample, the sample is analyzed with the MS and PTR processes to identify a susceptibility, resistance and/or pathogenicity marker(s) which has been linked with the identified or suspected microorganism. For example, in a sample known or suspected to contain S. aureus, the sample may be analyzed with the provided MS and PTR mass spectrometry processes to determine the presence or absence of penicillin binding protein 2a (PBP2a), the resistance marker associated with MRSA. In some embodiments, the provided MS and PTR mass spectrometry process detect intact proteins, such as PBP2a.

In some embodiments for predicting susceptibility of an identified microorganism or a microorganism of suspected identity in a sample, the sample is analyzed with the targeted MSⁿ processes to confirm or identify a susceptibility, resistance and/or pathogenicity marker(s) which has been linked with the identified or suspected microorganism. For example, in a sample known or suspected to contain S. aureus, the sample may be analyzed with the provided MSⁿ mass spectrometry procedures to confirm the presence or absence of PBP2a based on analysis of PBP2a fragment(s). Examples demonstrating the identification of PBP2a, KPC-2, and SHV-190 from clinical isolate lysates using the provided MSⁿ mass spectrometry procedures are shown herein.

In some embodiments, a sample of the microbial culture or biological sample that was used for testing in 1301 and 1302 (FIG. 13), is exposed for a brief period to conditions that induce antimicrobial resistance in the microorganism (1303). Following the induction step, the culture or sample may be analyzed for the presence of resistance markers (1304) via MS with PTR or via targeted MSⁿ mass spectrometry processes as described herein. A targeted search of resistance markers based on the typing information may be performed and the presence or absence or resistance markers reported (405). In some embodiments, the step of inducing resistance marker(s) (1303) is not necessary for the detection or confirmation of the presence or absence of the marker(s). Accordingly, induction of resistance markers is an optional step in the methods and workflows provided. As described herein, in some embodiments of the methods provided, the induction step of 1303 is not performed on the sample used for testing in 1301 and 1302, further analysis for the presence of resistance markers (1304) via MS with PTR or via MSⁿ mass spectrometry processes as described herein.

In some embodiments, susceptibility to the antimicrobial can also be determined or confirmed by analyzing the induced culture for markers indicative of microbial cell growth, growth inhibition, or cell death relative to an uninduced control culture. Alternatively, following a resistance induction step, a confirmatory test for antimicrobial susceptibility may be performed by growing the culture in the presence of a therapeutic concentration(s) of antimicrobial agent (1306). Susceptibility to the antimicrobial can be determined by analyzing the culture for markers indicative of disruption in normal growth, markers indicative of cell death and/or measuring cell death or substantial reduction in growth relative to a control lacking the antimicrobial. In some instances, a culture's failure to proliferate may first be identified by detecting expression of markers indicative of disruption in normal cell growth. Cells may display changes in markers indicative of disruption in normal cell growth within minutes of resistance induction whereas typical phenotypic test can take hours to determine whether culture growth is substantially reduced. Evidence of failure to proliferate, significant growth reduction, or cell death confirms the culture is susceptible. On the other hand, resistance to the antimicrobial can be determined by analyzing the culture for markers indicative of normal growth or division and/or measuring little or no cell death or reduction in growth relative to a control lacking the antimicrobial. Evidence of cell growth, division or proliferation in the presence of the antimicrobial confirms the culture is resistant.

In some embodiments, rapid phenotypic AST assays may be used to confirm the susceptibility predicted by the mass spectrometry methods provided herein. In some embodiments, a confirmatory test for antimicrobial susceptibility may be performed by verifying all resistance mechanisms known for the microbe are absent in the culture used to predict susceptibility (1302), or from a subculture. In other embodiments, following a resistance induction step, a confirmatory test for antimicrobial susceptibility may be performed by verifying all resistance mechanisms known for the microbe are absent in the induced culture (1307).

In some embodiments, prediction and/or confirmation of antimicrobial susceptibility by detection of resistance markers is preferred to a method which compares the growth of cultures incubated in the presence and absence of antimicrobial agent. Methods which compare growth in the presence and absence of antimicrobial agents after a brief period of incubation (for example, 4 hours) may fail to detect subpopulation(s) of resistant cells. However, resistance marker(s) from subpopulation(s) of resistant cells may be detectable by the mass spectrometry methods described herein.

Methods for inducing resistance markers in a microbial sample or culture are known in the art. Antimicrobial agents and a variety of environmental stress conditions can induce expression of antimicrobial resistance and other changes in the cell that impact innate antimicrobial susceptibility. For example, in some bacterial strains or types, inducible beta-lactamase production in bacteria can be induced by exposure of the cell to a beta-lactam agent. The action of the antibiotic on the cell wall activates a genetic cascade mechanism that initiates beta-lactamase production. In such bacteria strains or types, beta-lactamase production is significantly reduced or turned off when no antibiotic is present in or around the cell. Exposure of microbes to environmental conditions such as nutrient starvation/limitation (nutrient stress), reactive oxygen and nitrogen species (oxidative/nitrosative stress), membrane damage (envelope stress), elevated temperature (heat stress) and ribosome disruption (ribosomal stress) can initiate responses in the microbe that positively impact recruitment of resistance determinants or promote physiological changes that compromise susceptibility to a variety of antimicrobials. Poole (2012) J. Antimicrob. Chemother. Doi:10.1093/jac/dks196. For some microorganisms, pathogenicity and virulence markers can also be induced with appropriate inducer agents or culture conditions. In some embodiments, the inducing step is performed in a medium which supports cell replication. In some embodiments, the microbial sample is subjected to culture conditions to increase cell number while inducing expression of antimicrobial resistance. In certain embodiments, selective media can be used to allow clinically significant pathogen(s) to grow whilst suppressing the number of pathogenic strains.

In some embodiments, the microbial sample for induction is from blood, a blood culture, or from an agar plate. In some embodiments, the microbial sample, e.g., from blood, a blood culture, or an agar plate, is in log phase growth prior to or during resistance induction.

Generally, a sample containing the microbial isolate is first exposed to a level of an inducer insufficient to kill the microorganism but sufficient to induce resistance to one or more antimicrobials. For example, inducers can be one or more antimicrobial agents present at a low level (e.g., a sub-lethal level) or exposure to stress condition for a period of incubation. In some embodiments, the microbial sample can be exposed to antimicrobial at a concentration no greater than that is achievable at the site of infection, for example, below the resistance breakpoint. Antimicrobials that may be used to induce resistance include without limitation oxacillin, methicillin, ertapenem, vancomycin, and a fluoroquinolone antibiotic.

When performed in conjunction with the methods provided herein, the inducer exposure period may be brief, for example 120 minutes or less, since the presence of resistance markers are detected by mass spectrometry. The proteome of susceptible and resistant cells can show differences within minutes of being exposed to an antibiotic or other environmental stress condition.

In some embodiments, the microbial sample is exposed to antimicrobial resistance inducing conditions for about 60 minutes to about 120 minutes prior to resistance marker analysis. In some embodiments, the microbial sample is exposed to antimicrobial resistance inducing conditions for about 1 minute to about 60 minutes prior to resistance marker analysis. In some embodiments, the microbial sample is exposed to antimicrobial resistance inducing conditions for about 30 minutes to about 60 minutes prior to resistance marker analysis. In some embodiments, the microbial sample is exposed to antimicrobial resistance inducing conditions for about 1 minute to about 30 minutes prior to resistance marker analysis. In some embodiments, the microbial sample is exposed to antimicrobial resistance inducing conditions for about 20 minutes or less prior to resistance marker analysis. In other embodiments, the sample is exposed to inducing conditions for about 15 minutes or less prior to analysis. In still other embodiments, the sample is exposed to inducing conditions for about 10 minutes prior to analysis. In other embodiments, the sample is exposed to inducing conditions for about 5 minutes prior to analysis. In other embodiments, the sample is exposed to inducing conditions for about 3 minutes prior to analysis. In other embodiments, the sample is exposed to inducing conditions for about 1 minute prior to analysis.

Susceptibility reporting based solely on growth of an organism in the presence of different concentrations of antimicrobials can be inaccurate when organisms harbor certain resistance mechanisms. For example, isolates that produce carbapenemases such as SME and IMI often report susceptible in vitro testing to a range of cephalosporin antibiotics for which they should confer resistance in vivo. Clinical and Laboratory Standards Institute (CLSI), “Performance Standards for Antimicrobial Susceptibility Testing: Twenty-fifth Informational Supplement” MM100-S25 Vol 35, No 3 (2015). For this reason, it is common practice to edit such results when these resistance mechanisms are detected, irrespective of the growth based susceptibility results. Editing conventional phenotypic susceptibility based on resistance marker detection is still required but can be easily performed automatically using mass spectrometry as described.

As noted, in some embodiments, rapid phenotypic AST assays may be used to confirm the susceptibility predicted by the mass spectrometry methods provided herein. Rapid phenotypic AST assays are known in the art and are commercially available. Some rapid phenotypic AST assays are based on detection using flow cytometry. One rapid phenotypic method for AST is automated microscopy of immobilized individual live bacterial cells in which the properties of a small number of cells analyzed for a few hours in the presence of an antimicrobial are used to infer the phenotypic response of the entire bacterial culture. For example, the Accelerate ID/AST System (Accelerate Diagnostics, Inc.; Tucson, Ariz., USA) captures by time-lapse imaging the growth rate or growth inhibition of immobilized individual microbial cells in the presence of an antimicrobial agent and an algorithm converts of growth characteristics into a MIC value to the tested antimicrobial agent. A rapid phenotypic AST assay based on time-lapse tracking of bacterial growth in the presence of an antimicrobial is also available from QuantaMatrix, Inc. (Seoul, Korea).

Another rapid phenotypic AST method detects microbial growth in cultures incubated under various antibiotic conditions using electrochemical sensing of bacterial 16S rRNA (Liu et al. (2014) Ann Biomed Eng 42:2314-2321). An automated rapid phenotypic AST device available from Alifax (Padova, Italy) monitors bacterial growth based on light scattering with a customized antibiotic panel. All of these rapid phenotypic AST methods involve culturing the microbial sample for hours before susceptibility can be predicted since they rely on actual microbial growth.

The gold standard reference procedure for susceptibility testing requires at least a 16-24 hour incubation period for the tested culture. The rapid phenotypic AST assays reduce the incubation time to a matter of hours, in some cases 3-5 hours. However, certain resistance mechanisms detected by the gold standard reference procedure can be missed by the rapid methods which can lead to a false susceptible result.

Current standard susceptibility methods report 2-3 days after receipt of a specimen. For example, specimens such as blood typically require about a day of recovery and enrichment before plating onto agar. The specimen is then plated onto agar and examined the next day. Suitable colonies are removed from the agar to provide inoculum for identification and susceptibility testing. Typically, susceptibility testing requires about 5 to 24 hours incubation. Altogether, this standard methodology delays reporting 2-3 days. While waiting for the susceptibility result, an antibiogram can be checked to help guide selection of antimicrobial agents for empiric therapy. An antibiogram is a summary of previous susceptibility results (usually collected over the previous 12 months) that have been sorted by antibiotic, species and specimen information. The build-up of resistance makes the prediction of whether a particular species is likely to be susceptible or resistant to an antimicrobial less reliable because the prediction is based on a combination of susceptible and resistant results. For example, it is not possible to predict with any confidence when data collected over the previous 12 months shows 50% of isolates of a species are resistant. It may however be possible to distinguish between the sensitive and resistant isolates when results are summarized at a subspecies level which discriminates between different clones because a clone is much more likely to either be fully sensitive or resistant. The MS-PTR and MS' methods described herein provide a rapid, sensitive alternatives to make such determinations.

In another aspect, provided herein are methods for confirming or correcting antimicrobial susceptibility predictions or conclusions based on microbe identification or rapid phenotypic AST assays.

In some embodiments, methods are provided to confirm or correct an antimicrobial susceptibility prediction for a microbial or biological sample. In some embodiments, a targeted search by mass spectrometry is used to confirm or correct a rapid phenotypic AST result. In some embodiments, a microbe-containing sample with a predicted susceptibility status is subjected to a rapid mass spectrometry method in combination with PTR and targeted search for markers based on the predicted susceptibility. In other embodiments, the microbe-containing sample is briefly subjected to resistance-inducing conditions as described herein and then to a rapid mass spectrometry method in combination with PTR and targeted search for resistance or susceptibility markers and/or markers associated with cell growth, growth arrest, and/or cell death. In some embodiments, a microbe-containing sample with a predicted susceptibility status is subjected to a rapid mass spectrometry method in combination with MS' mass spectrometry processes and targeted search for markers based on the predicted susceptibility. In other embodiments, the microbe-containing sample is briefly subjected to resistance-inducing conditions as described herein and then to a rapid mass spectrometry method in combination with MSⁿ mass spectrometry processes and targeted search for resistance or susceptibility markers and/or markers associated with cell growth, growth arrest, and/or cell death. Results from targeted mass spectrometry search confirming the absence or presence of any predicted marker may be used to update a susceptibility report and/or database.

A known weakness of current AST systems, in particular rapid AST systems, is their inability to detect some resistance mechanisms without inclusion of additional screening tests or prolonged incubation. Resistances which are missed by rapid phenotypic AST procedures are well known. Detection errors of such AST procedures are reported, for example, as very major error (VME) (false-susceptible result of rapid AST) rates and major error (ME) (false-resistant result of rapid AST) for particular microorganism-antimicrobial combinations. The International Organization for Standardization (ISO) and the US Food and Drug Administration (FDA) define methods for calculating VME, ME, and other error rates of rapid AST, as well as acceptable levels of such rates. Rapid AST systems can perform poorly relative to ISO and FDA standards, especially for particular microorganism-antimicrobial combinations.

In some embodiments, microbe identification to the type or the clone level as described herein can be used to predict which resistances or resistance mechanisms may be missed by a rapid phenotypic AST method. A targeted search for the predicted resistances or resistance mechanisms can be performed by MS-PTR and/or MSⁿ processes and the susceptibility report corrected if the resistance markers are detected. In some embodiments, a database will list the resistance mechanisms responsible for the poor AST performance. A targeted search for the resistance mechanisms can be performed by MS-PTR and/or MSⁿ processes and the AST report corrected if the resistance markers are detected.

The present teachings are especially useful for the analysis and identification of intact proteins having molecular weight in excess of 50 kDa. Antimicrobial resistance markers include proteins which are of high molecular weight, such as in excess of 50 kDa. For example, the various penicillin binding proteins range in molecular weight from about 30 kDa to about 100 kDa, with many in excess of 50 kDa.

Use of PTR can improve high mass performance in mass spectrometry. In addition to improving the signal-to-noise ratios for this type of analysis, the reduction of charge on protein ions causes these large ions to refold in the gas phase. Without wishing to be bound by theory, it is believed that this more compact configuration reduces the collisional cross section of the protein ions and, accordingly, increases their stability against fragmentation by collision with background gas molecules present in the mass analyzer chamber during mass analysis in image current detection in an Orbitrap™ mass analyzer (a type of electrostatic mass analyzer commercially available from Thermo Fisher Scientific of Waltham, Mass., USA).

The various advantageous factors of mass spectrometry with PTR, at least including those described herein, can enable accurate identification of multiple intact proteins or large peptides from even very complex mixtures derived from microorganism cultures and biological samples. These processes lead to improved performance for detection of larger molecular weight resistance markers such as, for example, those derived from penicillin binding protein 2a (PBP2a) found in many MRSA clones. PBP2a has a molecular weight in the 78 k Da range.

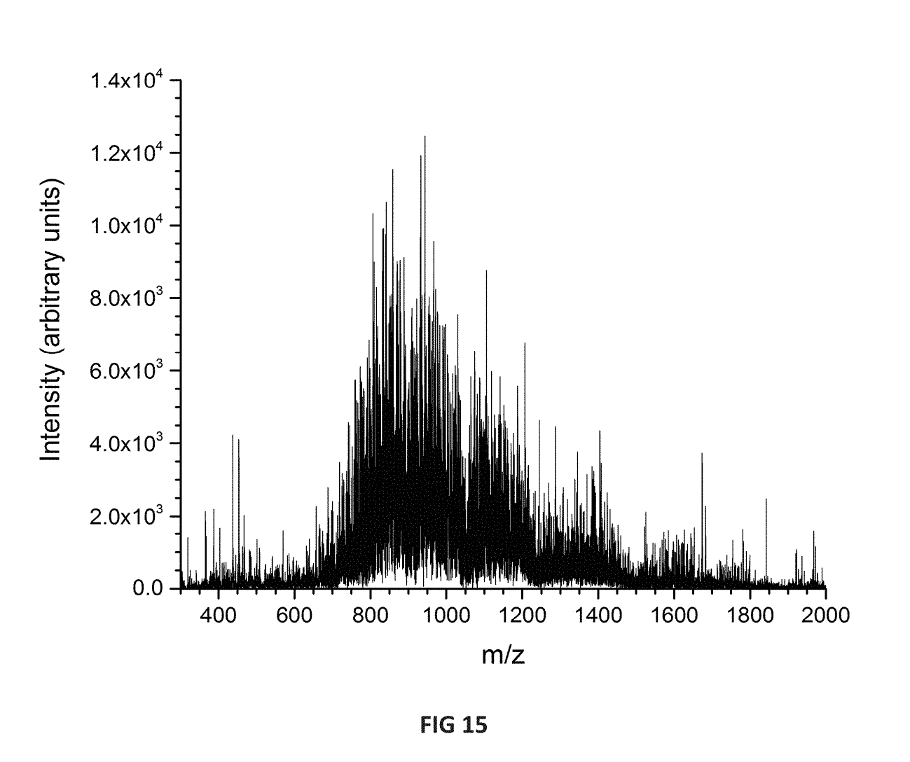

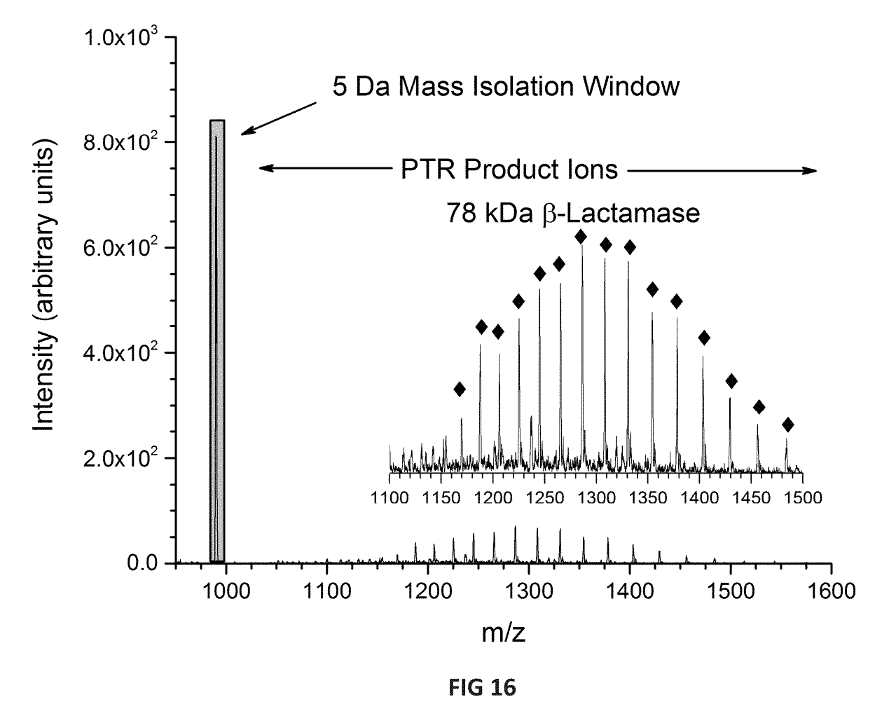

FIGS. 15 and 16 illustrate an example of analysis of beta-lactamase in partially purified protein extract from P. aeruginosa performed by the MS-PTR procedure provided herein. FIG. 15 depicts an electrospray ionization (ESI) mass spectrum generated from a partially purified protein extract from P. aeruginosa enriched for beta-lactamase. From this standard high resolution/accurate mass analysis no discernable peaks associated with the 78 kDa protein can be identified in this sample via single stage mass spectrometry. The only conclusion that can be drawn from this analysis is that the large number of overlapping protein peaks (with background noise) make it difficult identify individual components. FIG. 16 depicts a proton transfer reaction (PTR) product ion mass spectrum generated by isolating ions from the P. aeruginosa protein extract of FIG. 15 within a 5 Da mass window centered at m/z 990 and reacting the isolated ions with PTR reagent anions. Here the 78 kDa beta-lactamase is easily observed as the major component of the sample. The inset in FIG. 16 depicts the expanded view of the post-PTR charge states in the m/z 1100 to m/z 1500 range with the solid diamonds indicating the relevant charge states of the beta-lactamase. The analysis time associated with PTR was on the order of 5 ms.

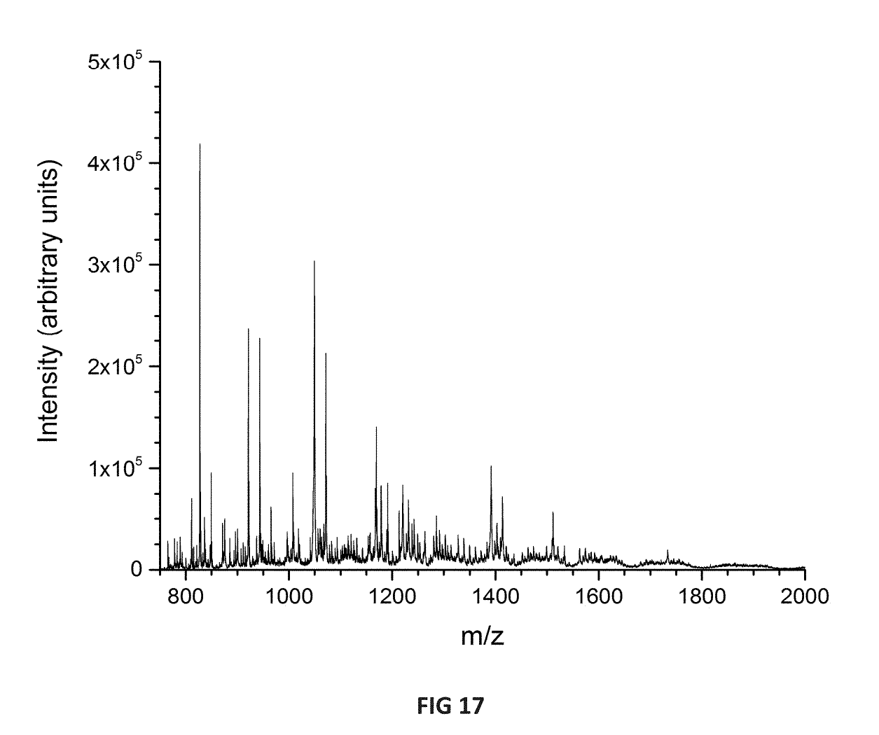

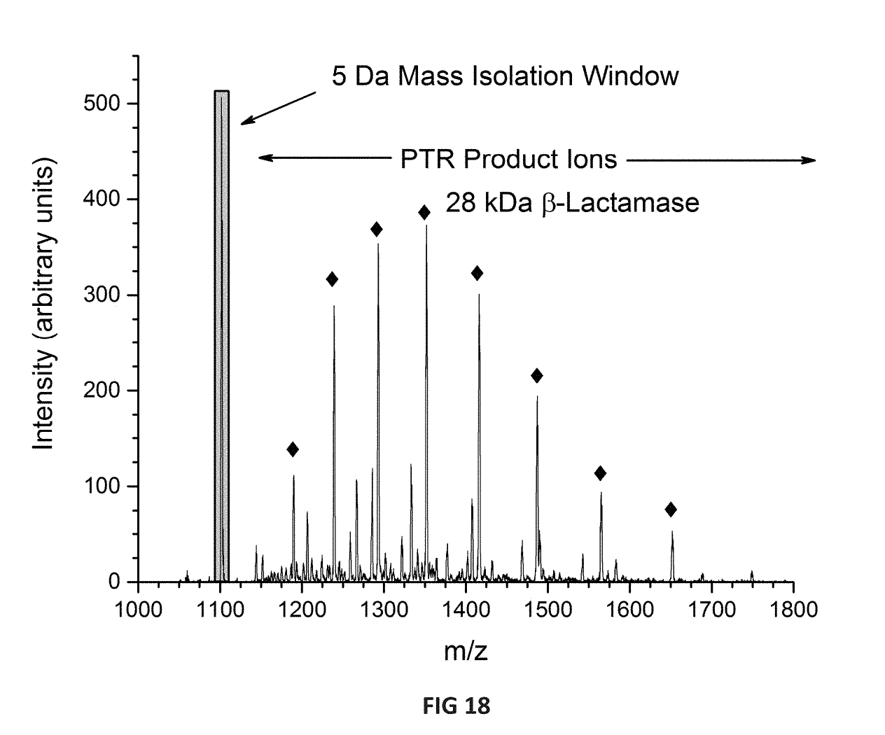

FIGS. 17 and 18 illustrate an example of analysis of beta-lactamase in partially purified protein extract from B. cereus performed by the MS-PTR procedure provided herein. FIG. 17 depicts the electrospray mass spectrum from a partially purified protein extract from B. cereus enriched for beta-lactamase. From this standard high resolution/accurate mass analysis no discernable peaks associated with the 28 kDa protein can be identified in the sample via single stage mass spectrometry. The only conclusion that can be drawn from this analysis is that the large number of overlapping protein peaks (with background noise) make it difficult identify individual components. FIG. 18 depicts the PTR product ion mass spectrum generated by isolating ions from the B. cereus protein extract of FIG. 17 within a 5 Da mass window centered at m/z 1101 and reacting the isolated ions with PTR reagent anions. Here the 28 kDa beta lactamase is easily observed as the major component of the sample with the solid diamonds indicating the relevant charge states of the beta-lactamase. The analysis time associated with PTR was on the order of 5 ms.

As discussed, in some cases of using mass spectrometry, it may be difficult due to low signal amounts, chemical background noise, or the presence of contaminating molecules, to confirm the presence of any given resistance marker for AST. In such cases, PTR can be used to effectively move the target resistance marker protein away from significant background noise.

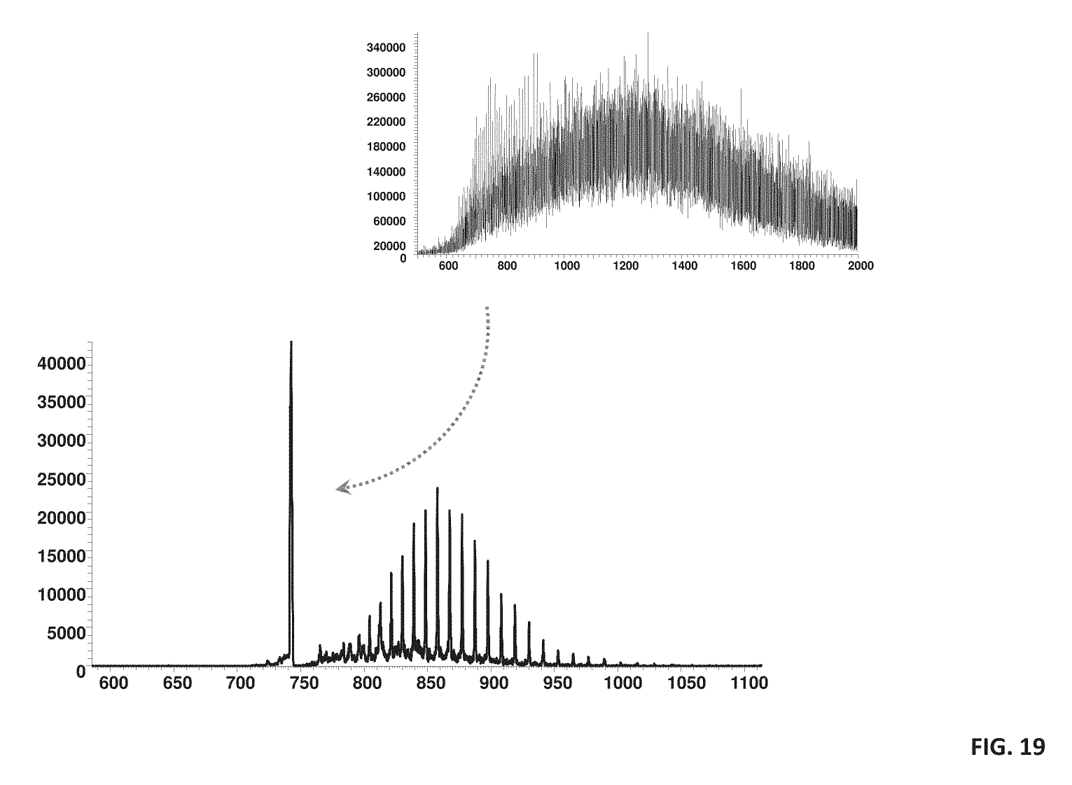

FIG. 19 illustrates an example of analysis of a recombinant PBP2a protein performed by the MS-PTR procedure provided herein. In the top section of FIG. 19 is shown the electrospray mass spectrum of the recombinant version of PBP2a generated in low resolution mode from a linear ion trap mass analyzer. After mass isolation of the target 5 Da mass isolation window at m/z 742.3 as shown, the isolated population of protein and background ions undergoes a PTR reaction for 1 ms and generates a second generation of ions free from chemical background noise and other contaminants (FIG. 19 bottom section). Therefore, the MS-PTR method separates the PBP2a protein signal from the background noise resulting in a clear charge-state distribution pattern that may be successfully used for identification of proteins in the sample.

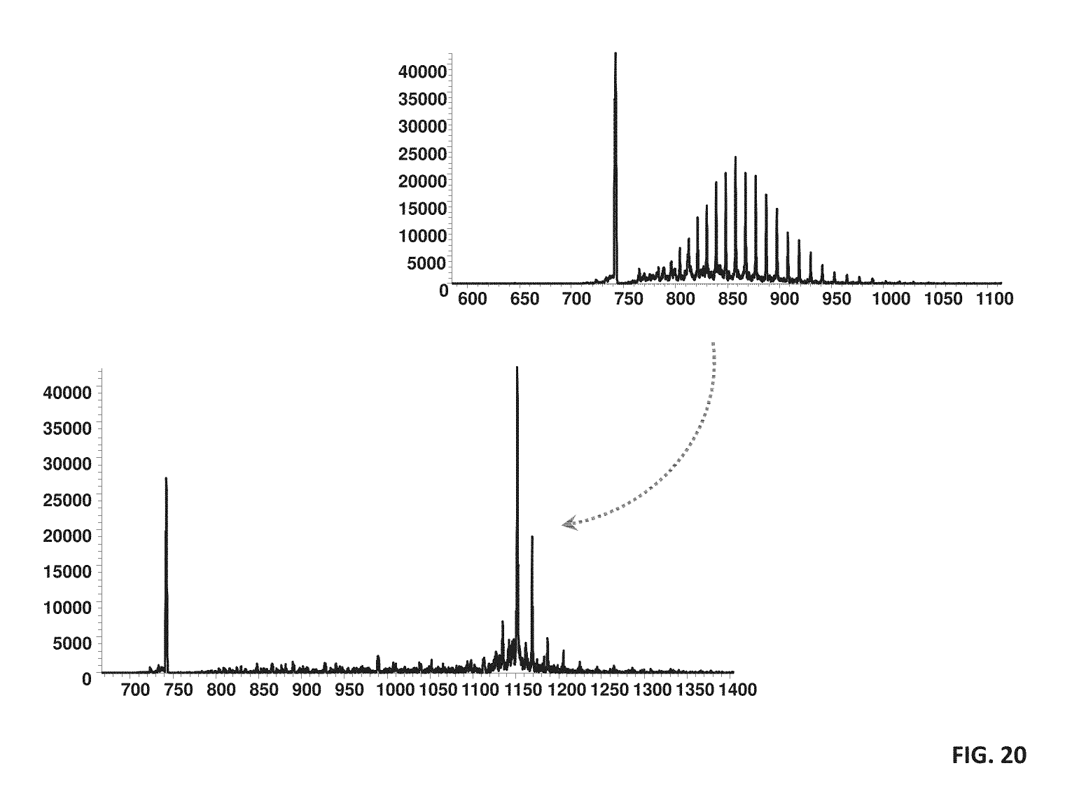

In the case where signal levels are low, the multiple charge states from PBP2a can be focused or concentrated into a signal of only 1 to 3 charge states to vastly improve detection limits for a the protein. FIG. 20 illustrates this focusing or ion concentration process using the ion parking techniques described herein. The top section of FIG. 20 shows mass isolation of the target 5 Da wide isolation window at m/z 742.3 with a 1 ms PTR reaction. The bottom section of FIG. 20 shows mass isolation of the target 5 Da wide isolation window at m/z 742.3 with ion parking at m/z 1134 target. As shown in FIG. 20, the ion parking technique concentrated the multiply-charges ion distribution generated via PTR (FIG. 20 top section) into primarily two charges states (FIG. 20 bottom section).

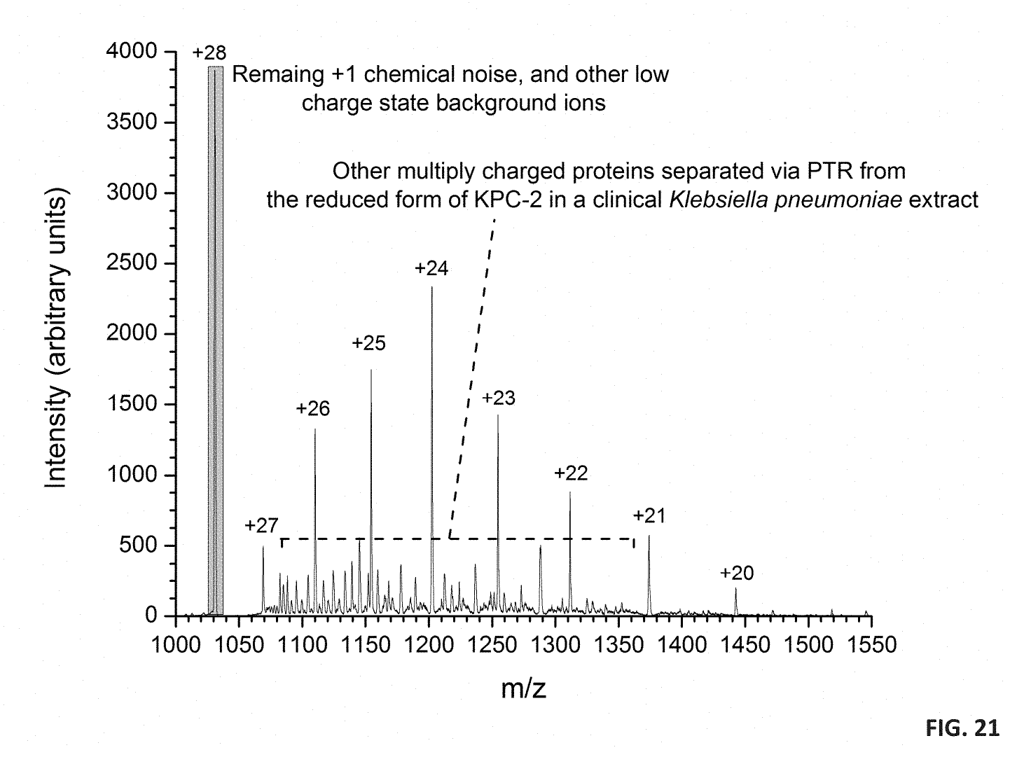

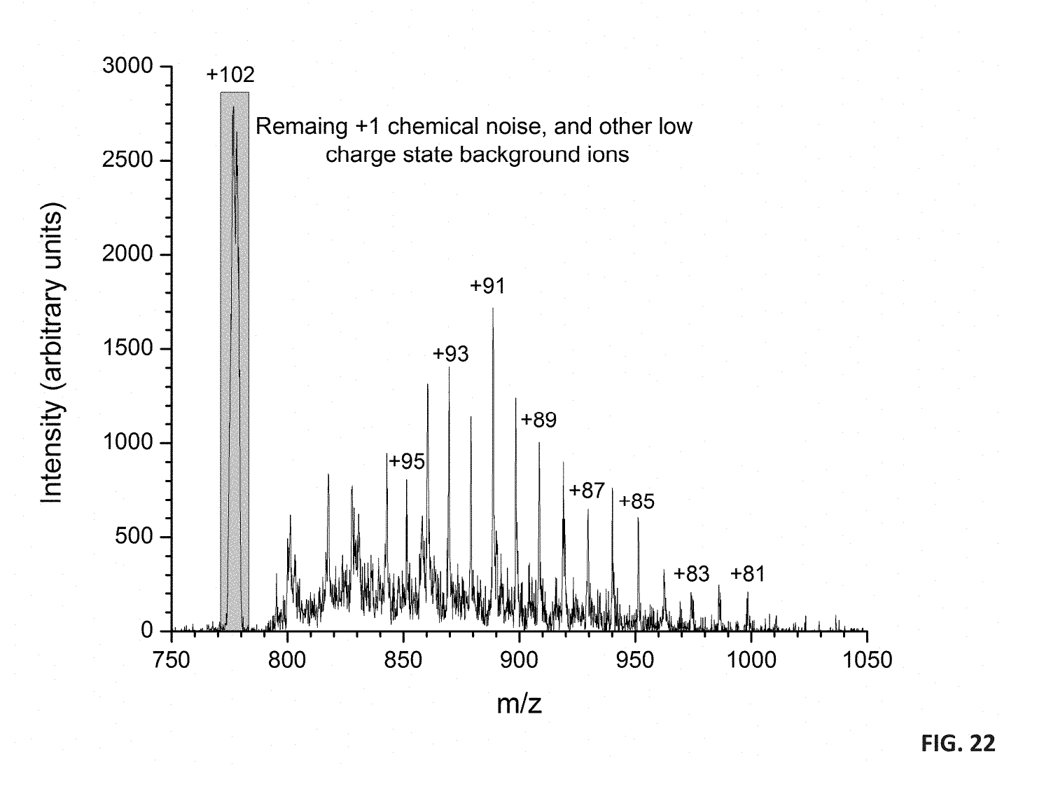

In some embodiments, the MS-PTR methods provided herein can be used to separate the target resistance marker from a variety of background and chemical noise related peaks in a rapid time frame. FIG. 21 illustrates an example of an analysis of a Klebsiella pneumoniae extract that is performed by the MS-PTR procedure provided herein. For this example, a clinical sample of K. pneumonia was cultured and an extract prepared. FIG. 21 depicts the PTR generated mass spectrum of the reduced and alkylated form of KPC-2 from the extract. Shown in FIG. 21 is the isolation of the +28 charge state derived from the reduced and alkylated version of KPC-2, followed by a PTR step for 2 ms. This procedure generated a cleaner mass spectrum showing the charge states ranging from +27 to +20 of KPC-2 and the subsequent separation of KPC-2 from the unwanted signals (see FIG. 21). FIG. 22 illustrates an example using a similar step for analysis of PBP2a from a clinical MRSA extract. For this example, a clinical sample of MRSA was cultured and an extract prepared. As shown in FIG. 22, the +102 charge state was isolated and reacted for 1 ms PTR to produce the mass spectrum ranging from the +95 to +81 charge state. This example demonstrates an enhanced signal for the PBP2a protein due to the separation of the protein from the background ions at 5 kDa window around m/z 775.

In some embodiments, ion parking techniques can be used to concentrate the signal from a wide variety different charge states (for example, of the order of 25) into a desired smaller number of charge states thus greatly improving detectability and signal-to-noise ratio. Identification to the species level along with typing restricts the list of possible markers for a targeted search to only those in the database aligned to the species and type. The target protein or polypeptide ion single species or multiple species may be chosen so as to be indicative, based on prior knowledge or information, either individually or in combination, of the presence in a sample of a specific resistance marker, pathogenicity marker, growth or no-growth marker, or indicator of cell apoptosis or death as a result of antimicrobial therapy.

As described herein, the systems and methods provided are applicable for targeted detection of antimicrobial resistance and susceptibility markers, microbial pathogenicity markers, for example but not limited to, toxins, hemolysin, and phenol-soluble modulin (PSM) protein family, and other markers of microbial activity, for example, factors indicative of cell growth, disruption of cell growth, no growth, and/or cell death. In some embodiments, the pathogenicity or virulence marker is detected is a Shiga (or Shigella) toxin. Shiga toxins are a family of toxin produced by Shigella dysenteriae and Entrohemorrhagic coli.

In some embodiments, provided are method for detecting microbial resistance markers from a microbial extract or lysate by performing MS-PTR and/or MSⁿ processes provided herein. In some embodiments, methods are provided for detecting beta-lactamases including without limitation SHV beta-lactamases and CTX-M beta-lactamases, KPC, and PBP2a.

The provided methods are applicable to substantially all microorganisms including, for example, Gram positive bacteria, Gram negative bacteria, mycobacteria, mycoplasma, yeasts, protozoans, and filamentous (i.e., microscopic) fungi.

Microorganisms possess a variety of mechanisms by which antimicrobial resistance is achieved. For example, common mechanisms by which microorganisms exhibit resistance to antimicrobials include without limitations antimicrobial inactivation or modification, cell wall or membrane impermeability, alteration of drug target site, overproduction of the antimicrobial target, alteration of metabolic pathways, change to porin activity, and efflux pumps that expel the antimicrobial agent from the cell before it can act on its target. In some cases, antimicrobial resistance may be the result of more than one mechanism. The various resistance mechanisms are often achieved through expression of particular proteins, metabolites, antibiotic modifications, lipids or carbohydrates directly or indirectly associated with the resistance phenotype. For example, enzymatic deactivation of beta-lactams in some penicillin-resistant bacteria occurs through the production of beta-lactamases which hydrolyze the active portion of the lactam. Vancomycin resistance is typically the enzymatic removal of the last two peptides of the cell wall peptide chains reducing its activity about 1000 times. Gram-negative bacteria may produce adenylating, phosphorylating or acetylating enzymes that modify an aminoglycoside so that it is no longer active as an antimicrobial agent.

In certain cases, alteration of porin channels in the outer membrane of bacteria can result in antimicrobial agents, e.g., beta lactam antibiotics, no longer allowed passage into the cell. In other examples, a resistance bacterium produces a modified protein or enzyme that binds to the antimicrobial agent, rendering it ineffective. For example, penicillin-binding proteins (PBP's) in both Gram-positive and Gram-negative bacteria may be altered through mutation so that beta-lactam antibiotics can no longer bind to them, leaving the cell resistant to these antibiotics. Fluoroquinolone resistance can be the result of mutations at particular sites in the drug's target enzymes.

In yet other instances, the microbe utilizes alternate pathways that bypass the metabolic pathway that the antimicrobial agent affects. For example, antimicrobial activity of sulfanamides is achieved through inhibition of the synthesis of folic acid early in the bacterial metabolic pathway. Some sulfonamide-resistant bacteria turn to utilizing preformed folic acid from a different pathway that is not affected by the sulfanamide. Some microbes reduce antimicrobial accumulation by increasing active efflux of the drugs across the cell surface. The resistant microbe can also reduce antimicrobial accumulation by decreasing the antimicrobial permeability into the cell and/or its interaction once inside. For example, efflux pumps are common mechanisms for ciprofloxacin and silver resistances.

Exemplary antimicrobial resistance markers include, without limitation, Class A beta lactamases, extended-spectrum beta-lactamases (ESBL), TEM beta-lactamases, SHV beta-lactamases, CTX-M beta-lactamases, OXA beta-lactamases, PER beta-lactamases, VEB beta-lactamases, GES beta-lactamases, IBC beta-lactamases, AmpC-type beta-lactamases, inhibitor-resistant TEM β-lactamases, carbapenemases, imipenemase (IMP), Verona intron-encoded metallo-beta-lactamase (VIM), Klebsiella pneumoniae carbapenemase (KPC), CMY carbapenemases, New Delhi metallo-beta-lactamase (NDM), outer membrane porin OprD, aminoglycoside-modifying enzyme, and penicillin-binding proteins, including without limitation PBP2a and PBP5.

Microbial resistances of particular clinical interest include MRSA, vancomycin-resistance enterococci (VRE) such as E. faecalis, Enterobacteriaceae fluoroquinoline resistance, and Pseudomonas resistance due to porin/efflux expression. In certain embodiments, antimicrobial resistance markers detected with the provided methods include KPC carbapenemase, KPC, ESBL, PBP2a, colistin resistance, and markers for VRE.

Dominant microbial clones often combine resistance to multiple antimicrobials with the ability to outcompete other strains. For example, 90% of Asian methicillin resistant MRSA outbreaks have been due to clone ST139. Approximately 51% of E. coli community acquired infections and 67% of hospital acquired infections in a single state in the US are due to six clones. Also 60%-80% of fluoroquinoline-resistant isolates and 50 to 60% of ESBL-producing isolates of ExPEC Escherichia coli are globally accounted for by MLST type ST131. Klebsiella pneumoniae MLST type ST258 is endemic to the USA, Israel, Greece, Italy, Poland and Columbia. Escherichia coli MLST types ST131, ST69, ST73 and ST95 account for 45% of ExPEC strains from community and hospital urine samples recovered in 2007-2009 and 2007-2008 in the Northwest and the East Midlands of England respectively, as well as 58% of those bacteraemia in Northern England from 2010-2012.

The systems and methods provided herein are applicable to analyzing samples containing microorganisms including, but not limited to, pure or mixed microbial cultures, samples containing a single microorganism type or mixtures of microorganisms, blood culture samples, direct clinical samples (e.g., surface swabs, bodily fluids, etc.), and cultured clinical samples. The sample may be of any type suspected to contain one or more microorganisms including, without limitation, isolated colonies from a culture plate, including without limitation a blood agar plate and a culture plate containing an inducer of antimicrobial resistance; cells from liquid growth medium including without limitation a blood culture and a liquid growth medium containing an inducer of antimicrobial resistance; blood, saliva, urine, stool, sputum, wound and body site swabs; food and beverage; soil, water, air; environmental and industrial surface swabs. Specimens from normally sterile body sites usually contain a single microbial pathogen. The method of identification provided herein is capable of discriminating a single pathogen from a mixed pathogen. In the event of a single microbial pathogen, susceptibility testing can be initiated without incurring a delay from having to first subculture with results available, for example, within a day of receipt of the specimen. In some embodiments, antimicrobial susceptibility results may be obtained in about two hours of the start of the provided method. In some embodiments, antimicrobial susceptibility results may be obtained in about an hour of the start of the provided method.

For samples containing a mixture of microorganisms or for a mixed microbial culture, methods provided may detect one or more resistance markers expressed by the microbes identified in the mixture. In some cases, for example, two different pathogens may be identified and two different sequences for resistance markers may be detected. Strain information may then permit assigning a resistance to a particular pathogen. In other cases, for example, two different pathogens are identified but the pathogens are of a type for which the resistance marker sequences are the same. In such cases, the mass spectrometry methods using MS-PTR and/or MS' provided herein for detection of secondary information, such as other markers expressed on a resistance marker-bearing plasmid, can be used to disambiguate the resistance origin.

Methods in accordance with the present teachings may comprise at least two or more of the following steps: microbial cell disruption, solubilization of proteins, sample clean-up (to desalt, remove insoluble components and debris, and/or concentrate), sample infusion or flow injection, fast partial liquid chromatographic separation, size exclusion chromatography, standard chromatographic separations (reverse and normal phase), isoelectric focusing, emerging LC techniques such as digital LC or related approaches, ionization of proteins in solution, isolation of a given m/z range of the ions, causing the isolated range of ions to undergo PTR so as to form first-generation PTR product ions, optional isolation of an m/z range of the first-generation PTR product ions, optional mass spectrometry in MS or MS/MS mode, optionally causing the isolated range of first-generation PTR product ions to undergo a second PTR reaction so as to form second-generation PTR product ions, mass spectrometry in MS or MS/MS mode, and microbial identification via molecular weight analysis, protein sequence analysis, and/or spectral analysis using a variety of different statistical approaches. Preferably, but not necessarily, the mass spectrometry steps are performed with a high-resolution, accurate mass instrument, such as a mass spectrometer comprising an Orbitrap™ mass analyzer. Some embodiments further include incubation of the microbial sample in one or more concentrations of an antibiotic prior to start of the mass spectrometry AST assay. Some embodiments include incubation of the microbial sample under a stress condition prior to start of the mass spectrometry AST assay.

In the provided methods and systems, microbial cells in a sample are disrupted or lysed to form a microbial lysate or extract. Disruption of bacterial cells, fungal cells, mycoplasma cells, and the like may be achieved by mechanical, chemical, enzymatic and other means as are commonly known in the art. Mechanical approaches include bead beating, use of pressure such as from a French press and the like, sonication or other methods known in the art. Chemical methods include exposure to chaotropes such as urea, thiourea, or guanidine HCl to lyse the microbial cells and solubilize their contents. Alternatively, organic acid/solvents mixtures may be utilized to disrupt cells. Enzymatic methods include using lysozyme, lysostaphin or other lytic enzymes to form “holes” in the bacterial cell walls that allow the contents to leak out into the surrounding solution.

Sample clean-up steps prior to analysis by mass spectrometry include, without limitation, procedures that remove salts or lipids from the crude cell lysate or extract and procedures that enrich one or more analytes of interest relative to one or more other components of the sample. In some embodiments, sample clean-up or sample purification may refer to sample processing and clean-up in a separate laboratory that has biosafety level-three facilities for handling mycobacteria or filamentous fungi. In some embodiments, sample purification or sample clean-up may be accomplished by a solid phase extraction device, in-line size exclusion chromatography and/or an optional chromatography column.

In one embodiment, a sample-purification device may include a solid phase extraction (SPE) cartridge. In some embodiments, the SPE cartridge may be in line directly with the high resolution/accurate mass instrument. In one embodiment, the SPE cartridge may be a polypropylene tip with a small volume of silica or other sorbent containing bonded C4, C8, C18, RP4H, or RPSH or other functional groups immobilized in the cartridge, for example, a StageTip™ cartridge (Thermo Fisher Scientific). In alternative embodiments, polymeric sorbents or chelating agents may be used. The bed volume may be as small as 1 μL or less but greater volumes may also be used. In some embodiments of the mass spectrometry system and method provided, each SPE cartridge is used only once, minimizing carryover problems from one microbial cell sample to another.

In one embodiment, a sample-purification device may be an in-line size exclusion chromatography column designed to remove unwanted salts, small molecules, and lipids from the microbial lysate or extract. The approach can be used to separate medium and large molecular weight proteins as well. Phases are selected to be compatible with partial (i.e., less than 100 percent) organic solutions and organic acids. Phases can accommodate protein size distributions that differ in molecular weight from 10³ to 10⁸ Da. Flow rates can be adjusted in real time to effect separation of intact proteins from small molecules with separation flow rates typically much less than the higher flow rates used to remove small molecules, lipids, and salts from the system. In some embodiments, a sample-purification device may also be heated to facilitate faster diffusion rates for intact proteins, thus significantly shortening run times. The flow of mobile phase through a sample-purification device may also be diverted during a portion of the clean-up process to remove certain impurities from the flow stream and prevent them from entering the mass spectrometer.

In one embodiment, the optional chromatography column may include a column configured for at least partial chromatographic separation of the proteins in the sample. The stationary phase in the chromatography column may be porous or non-porous silica or agarose particles, or a monolithic material polymerized or otherwise formed inside the column. The stationary phase may be coated with an appropriate material such as C18, C8, C4 or another suitable derivative, or contain cation exchanger or other material, or the combination of the above to facilitate the separation of the proteins, and such material may be chemically bonded to the particles or monolith inside the column. Particle sizes typically range from about 1.5 μm to 30 μm. Pore sizes can range from 50 to 300 angstroms. Inside diameters of columns typically range from about 50 μm to 2.1 mm, and column length from about 0.5 cm to 25 cm, or other. In some embodiments, the mobile phase or eluent may be a pure solvent, or a mixture of two or more solvents, and may contain added salts, acids and/or other chemical modifiers. In some embodiments, the proteins are separated on the column based on one or more physiochemical properties, including size, net charge, hydrophobicity, affinity, or other physiochemical properties. Chromatographic separation methods include one or more of ion exchange, size exclusion, hydrophobic liquid interaction chromatography (HILIC), hydrophobic interaction, affinity, normal-phase, or reverse-phase chromatography.

Additional methods of purifying the samples may include, without limitation, liquid chromatography, HPLC, UHPLC, precipitation, solid-phase extraction, liquid-liquid extraction, dialysis, affinity capture, electrophoresis, filtration, ultra-filtration or other suitable methods known in the art for purification.

In some embodiments of the methods provided for predicting and/or confirming antimicrobial susceptibility, the required time for the analysis may be shortened by employing either a SPE step, a time-compressed chromatography step as described, for example, in U.S. Pat. No. 5,175,430, or the method of Fast Partial Chromatographic Separation (FPCS) in the chromatography step as described in U.S. Pat. No. 9,074,236. Generally, in performing FPCS, a crude extract of microbial cells containing a complex mixture of various organic and inorganic analytes (small organic molecules, proteins and their naturally occurring fragments, lipids, nucleic acids, polysaccharides, lipoproteins, etc.) is loaded on a chromatographic column and subjected to chromatography. However, instead of allowing a gradient to elute each analyte separately (ideally, one analyte per chromatographic peak), the gradient is intentionally accelerated to the extent that substantially no chromatographic peaks obtained, for example, approximately eight minutes or less, and preferably five minutes or less instead of a much longer run time that would be required to obtain a baseline separation. In the FPCS separation, many analytes are intentionally co-eluted from the column at any given time according to their properties and the type of chromatography (reverse phase, HILIC, etc.) used. Overall, FPCS-MS provides rapid analysis, maximizing the number of samples that can be analyzed in a set period of time, while providing the necessary information about the sample. In other embodiments, partial or incomplete separation may be also accomplished by other methods.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following examples, which should not be construed as limiting the scope of the teachings in any way. Any patents, patent publications or technical publications or technical documents mentioned within this disclosure are hereby incorporated by reference herein. If any statements in the mentioned documents should conflict with statements made in this application, then the present disclosure will control.

1. A method for predicting antimicrobial susceptibility of a microbe in a sample, the method comprising:
a) disrupting one or more microbes present in a sample to form a fluid microbial extract;
b) separating a soluble protein fraction from an insoluble protein fraction present in the fluid microbial extract to form a liquid solution of the soluble protein fraction;
c) ionizing a stream or flow of the solution of soluble protein fraction to form one or more ionized proteins, wherein the one or more ionized proteins comprises positively charged ions, the positively charged ions comprising a plurality of ion species;
d) analyzing the plurality of ion species with a mass analyzer of a mass spectrometer system to obtain mass spectral data, searching a database containing mass spectral data of known microbial proteins, and identifying at least one microbe from the database based on the determined mass spectral data of the ion species derived from ionization of the proteins from the sample;
e) from a database containing molecular weights and/or sequence data of the analyte compounds associated with antimicrobial susceptibility, selecting a list of analyte compounds whose presence in the identified microbe is predictive of the state of antimicrobial susceptibility for the identified microbe to form a selected analyte database, the list of analyte compounds comprising protein and/or polypeptide compounds;
f) isolating at least a first subset of the plurality of ion species with the mass spectrometer system, each isolated subset of the at least first isolated subset comprising a respective single mass-to-charge (m/z) ratio or range of m/z ratios, wherein the at least first isolated subset is selected based on the microbe identification and the selected analyte database;
g) generating a plurality of first-generation product ions species from each isolated subset of ion species by causing each said isolated subset of ion species to be reacted with reagent anions that, upon reaction, extract protons from each of one or more ion species of said isolated subset of ion species that comprises a protonated molecule of a protein or polypeptide compound whose charge is reduced by the reaction;
h) acquiring at least one mass spectrum using the mass spectrometer system either of some or all of the first-generation product ion species or of a plurality of second-generation product ion species generated by further reaction or fragmentation of the first-generation product ion species;
i) using m/z ratios of the first-generation or second-generation product ion species to search the selected analyte database; and
j) identifying the presence or absence of at least one analyte compound from the sample predictive of the state of antimicrobial susceptibility for the identified microbe.

2. The method of claim 1, wherein the analyzing in (d) comprises:
i) acquiring one or more mass spectra representative of the plurality of ion species;
ii) determining molecular weights for the proteins from the one or more mass spectra; and
iii) using the determined molecular weights to search a database containing molecular weights of known microbial proteins and identifying at least one microbe from the database based on the basis of the determined molecular weights of the proteins from the sample.

3. The method of claim 1, wherein the analyzing in (d) comprises:
i) in a first mass spectrometry step, acquiring one or more first mass spectra representative of one or more of the plurality of ion species;
ii) determining molecular weights for the proteins from the one or more first mass spectra;
iii) using the determined molecular weights to search a database containing molecular weights of known microbial proteins, and selecting a subset of candidate microbes from the database;
iv) in a second mass spectrometry step, selecting one or more precursor ions of the proteins from the of the plurality of ion species and fragmenting the precursor ions by fragmentation means to produce a plurality of MS² product ions;
v) using m/z ratios of the one or more precursor ions and/or the plurality of MS² product ions to search a database containing molecular weights of known microbial proteins and product ion m/z values of the known microbial proteins; and identifying at least one microbe from the database based on the basis of the determined molecular weights and product ion m/z values of the ionized proteins from the sample.

4. The method of claim 1, wherein the analyzing in (d) comprises:
i) acquiring one or more mass spectra representative of one or more of the plurality of ion species; and
ii) using the acquired mass spectra and a mass spectral deconvolution program that differentiates signals of the mass spectra to search a database containing mass spectra of known microbial proteins and identifying at least one microbe from the database based on the basis of the acquired mass spectra of the ionized proteins from the sample.

5. The method of claim 1, wherein the generating of the plurality of first-generation product ions species from each isolated subset of ion species in (g) is followed by an ion fragmentation reaction of the PTR product species to obtain a plurality of fragment ion species.

6. The method of claim 1, wherein the microbe is identified as a particular sub-species of a microorganism and the list of analyte compounds includes those that are predictive of the state of antimicrobial susceptibility for the particular subspecies.

7. (canceled)

8. The method of claim 1, wherein step b) is performed using a solid phase extraction device.

9. The method of claim 1, wherein (a)-(j) are performed using automated instrumentation.

10. A method for predicting antimicrobial susceptibility of a microbe in a sample, the method comprising:
a) exposing a sample containing one or more microbes to culture conditions which can induce production of antimicrobial resistance markers to form an induced sample;
b) disrupting one or more microbes present the induced sample to form a fluid extract;
c) separating a soluble protein fraction from an insoluble protein fraction present in the fluid extract to form a liquid solution of the soluble protein fraction;
d) ionizing a stream or flow of the solution of soluble protein fraction to form one or more ionized proteins, wherein the one or more ionized proteins comprises positively charged ions, the positively charged ion comprising a plurality of ion species;
e) analyzing the plurality of ion species with a mass analyzer of a mass spectrometer system to obtain mass spectral data, searching a database containing mass spectral data of known microbial proteins, and identifying at least one microbe from the database based on the determined mass spectral data of the ion species derived from ionization of the proteins from the induced sample;
f) from a database containing molecular weights of antimicrobial resistance markers expressed by microbes, selecting a list of antimicrobial resistance markers associated with the identified microbe species to form a selected marker database, the list of antimicrobial resistance markers comprising protein and/or polypeptide compounds;
g) isolating at least a first subset of the plurality of ion species with the mass spectrometer system, each isolated subset of the at least first isolated subset comprising a respective single mass-to-charge (m/z) ratio or range or m/z ratios, wherein the at least first isolated subset is selected based on the microbe identification and the selected marker database;
h) wherein the at least first isolated subset of ions are reduced in charge state by: i) at least one proton transfer reaction (PTR) to obtain a plurality of PTR product ion species, or ii) at least one PTR to obtain a plurality of PTR product ion species followed by an ion fragmentation reaction of the PTR product ion species to obtain a plurality of fragment ion species;
i) acquiring at least one mass spectrum using the mass spectrometer system of at least some of the PTR product ion species or at least some of the fragment ion species;
j) using m/z ratios of the PTR product ion species or the fragment ion species to search the selected marker database; and
k) determining the presence or absence of at least one antimicrobial resistance marker from the induced sample, wherein the presence or absence of the identified antimicrobial resistance marker in the induced sample as compared to the microbial sample not induced is predictive of antimicrobial susceptibility for the identified microbe.

11. The method of claim 10, wherein the exposing to culture conditions which can induce production of antimicrobial resistance markers is for about 1 minute to about 30 minutes.

12. The method of claim 10, wherein the exposing comprises contacting the sample with at least one antimicrobial agent with a concentration below the resistance breakpoint for the at least one antimicrobial agent.

13. The method of claim 10, wherein the analyzing in (e) comprises:
i) acquiring one or more mass spectra representative of the plurality of ion species;
ii) determining molecular weights for the proteins from the one or more mass spectra; and
iii) using the determined molecular weights to search a database containing molecular weights of known microbial proteins and identifying at least one microbe from the database based on the basis of the determined molecular weights of the proteins from the induced sample.

14. The method of claim 10, wherein the analyzing in (e) comprises:
i) in a first mass spectrometry step, acquiring one or more first mass spectra representative of one or more of the plurality of ion species;
ii) determining molecular weights for the proteins from the one or more first mass spectra;
iii) using the determined molecular weights to search a database containing molecular weights of known microbial proteins, and selecting a subset of candidate microbes from the database;
iv) in a second mass spectrometry step, selecting one or more precursor ions of the proteins from the of the plurality of ion species and fragmenting the precursor ions by fragmentation means to produce a plurality of MS2 product ions;
v) using m/z ratios of the one or more precursor ions and/or the plurality of MS2 product ions to search a database containing molecular weights of known microbial proteins and product ion m/z values of the known microbial proteins; and identifying at least one microbe from the database based on the basis of the determined molecular weights and product ion m/z values of the ionized proteins from the induced sample.

15. The method of claim 10, wherein the analyzing in (e) comprises:
i) acquiring one or more mass spectra representative of one or more of the plurality of ion species; and
ii) using the acquired mass spectra and a mass spectral deconvolution program that differentiates signals of the mass spectra to search a database containing mass spectra of known microbial proteins and identifying at least one microbe from the database based on the basis of the acquired mass spectra of the ionized proteins from the induced sample.

16. The method of claim 10, wherein the microbe is identified as a particular sub-species of a microorganism and the list of antimicrobial resistance markers includes those that are predictive of the state of antimicrobial susceptibility for the particular sub-species.

17-19. (canceled)

20. A method for confirming a predicted antimicrobial susceptibility of an identified microbe in a sample, the method comprising:
a) exposing a sample containing an identified microbe with a predicted antimicrobial susceptibility to culture conditions which can induce production of antimicrobial resistance markers to form an induced sample;
b) extracting, from the induced sample, a liquid solution comprising a mixture of sample-derived proteins and polypeptides;
c) introducing at least a first portion of the liquid solution into an ionization source of a mass spectrometer;
d) generating positively charged ions of the mixture of sample-derived proteins and polypeptides, the positively charged ions comprising a plurality of ion species;
e) isolating at least a first subset of the plurality of ion species, each isolated subset of the at least a first isolated subset comprising a respective single mass-to-charge (m/z) ratio or range of m/z ratios;
f) generating a plurality of first-generation product ions species from each isolated subset of ion species by causing each said isolated subset of ion species to be reacted with reagent anions that, upon reaction, extract protons from each of one or more ion species of said isolated subset of ion species that comprises a protonated molecule of a protein or polypeptide compound whose charge is reduced by the reaction;
g) acquiring at least one mass spectrum using a mass analyzer of the mass spectrometer, either of some or all of the first generation product ion species or of a plurality of second-generation product ion species generated by further reaction or fragmentation of the first-generation product ion species;
h) conducting a search of the at least one mass spectrum of either first-generation or second-generation product ion species for a set of one or more m/z ratios that are diagnostic of at least one marker for the predicted antimicrobial susceptibility of the identified microbe;
i) determining the presence or absence of at least one antimicrobial susceptibility marker from the induced sample; wherein presence of an antimicrobial susceptibility marker in the induced sample as compared to the microbial sample not induced confirms the predicted antimicrobial susceptibility of the identified microbe.

21. (canceled)

22. The method of claim 20, wherein the exposing comprises contacting the sample with at least one antimicrobial agent with a concentration below the resistance breakpoint for the at least one antimicrobial agent.

23. (canceled)

24. The method of claim 20, wherein a rapid phenotypic antimicrobial susceptibility test was used to predict the antimicrobial susceptibility of the identified microbe prior to (a).

25. The method of claim 20, further comprising separating a soluble protein fraction from an insoluble protein fraction present in the liquid solution of (b) prior to the introducing of step (c).

26. The method of claim 25, wherein the separating of the soluble protein fraction is performed using a solid phase extraction device.

27-31. (canceled)
